(19) United States
(12) Patent Application Publication
(10) Pub. No.: US 2022/0288225 A1
(43) Pub. Date: Sep. 15, 2022

(54) MATERIALS AND METHODS FOR ACTIVATING ANTIGEN-SPECIFIC T CELL RESPONSES

(71) Applicant: NORTHWESTERN UNIVERSITY, Evanston, IL (US)

(72) Inventor: Jennifer D. Wu, Wilmette, IL (US)

(21) Appl. No.: 17/635,444

(22) PCT Filed: Aug. 21, 2020
(86) PCT No.: PCT/US20/47470

§ 371 Date: Feb. 15, 2022

Related U.S. Application Data
(60) Provisional application No. 62/890,933, filed on Aug. 23, 2019.

(51) Int. Cl. A61K 47/68; A61K 39/395; A61P 35/00

(52) U.S. Cl. CPC A61K 47/6849; A61K 47/6811; A61K 39/3955; A61P 35/00

(57) ABSTRACT

Described herein are Natural Killer Group 2D (NKG2D) agonist complexes comprising a soluble MHC I Chain-related molecule (sMIC) and a non-blocking sMIC-neutralizing antibody. Methods for activating CD8 T cells and methods for treating MIC-negative cancers and viral infections using such complexes are also provided.

### CROSS-REFERENCE TO RELATED APPLICATIONS

The present application claims the benefit of priority to U.S. Provisional Application No. 62/890,933, filed Aug. 23, 2019, the disclosure of which is incorporated by reference in its entirety.

### STATEMENT OF GOVERNMENT SUPPORT

This invention was made with government support under R01CA208246 and R41CA206688-01A1 awarded by the National Institute of Health (NIH). The government has certain rights in the invention.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: Filename: 2019-146_Seqlisting.txt; Size: 75,481 bytes; Created: Aug. 21, 2020.

### BACKGROUND

Effective T cell co-stimulation is critical for the primary induction and subsequent maintenance of antigen-specific T cell responses. In addition to increased co-inhibitory signals, insufficient co-stimulatory tumor microenvironment accounts for a great deal of the suboptimal activation and maintenance of tumor-killing CD8 T cells. Thus, one of the major goals in the immunotherapy of cancer is to provide sustainable co-stimulatory signal to empower the generation and persistence of effective tumor-killing CD8 T cells and ultimately to achieve durable tumor control. Yet, beyond engineered CAR-T cells that contain co-stimulatory motif in the engineered TCR, means to empower sustained in situ CD8 T cell co-stimulation are still far from expectations, due to the unsustainable expression of the canonical and activation-induced family of co-stimulatory receptors on CD8 T cells in the tumor microenvironment.

### BACKGROUND

Effective T cell co-stimulation is critical for the primary induction and subsequent maintenance of antigen-specific T cell responses¹⁻³. Insufficient co-stimulation accounts for a great deal of the suboptimal activation and maintenance of tumor-killing antigen-specific CD8 T cells^{2,3}. Thus, one of the major goals in the immunotherapy of cancer is to provide sustainable co-stimulatory signal to empower the generation and persistence of effective tumor-killing CD8 T cells and ultimately to achieve durable tumor control. Yet, beyond engineered CAR-T cells that contain co-stimulatory motif in the engineered TCR, means to empower sustained in situ CD8 T cell co-stimulation are still far from expectations, due to: 1) unsustainable expression of the canonical and activation-induced family of co-stimulatory receptors on CD8 T cells in the tumor microenvironment; 2) considerable autoimmune cytotoxicity potentially resulted from unwanted co-stimulation of lymphocytes than cytotoxic T cells⁴.

Natural Killer Group 2D (NKG2D), an activating receptor expressed by all human NK cells, is also defined as a co-stimulatory receptor for human NKT, CD8T and γβT cells⁵⁻¹⁰. Similar to the canonical co-stimulatory molecule CD28 and activation-induced TNF-R superfamily of costimulatory molecules, NKG2D co-stimulation amplifies the magnitude of CD3/TCR signaling. Different from these well-studied co-stimulatory molecules, expression of NKG2D expression is independent of T cell activation or functional status and also not found on CD4 T cells or B cells under normal condition⁵⁻¹¹. Compelling evidence has demonstrated that NKG2D co-stimulation can not only bolster CD8 T cell effector function, but is also important for memory CD8 T cell development and rescue¹¹⁻¹³. These understandings have endorsed NKG2D as an instrumental co-stimulatory molecule to generate effective and persistent tumor-killing antigen-specific CD8 T cells.

### SUMMARY

In one aspect, described herein is Natural Killer Group 2D (NKG2D) complex comprising a soluble MHC I chain-related molecule (sMIC) and a non-blocking sMIC-neutralizing antibody. In some embodiments, the non-blocking antibody in the complex comprises CDRs set forth in SEQ ID NOs: 4-9. In some embodiments, the non-blocking antibody in the complex comprises a light chain variable region set forth in the SEQ ID NO: 11. In some embodiments, the non-blocking antibody in the complex comprises a heavy chain variable region set for in the SEQ ID NO: 10. In some embodiments, the non-blocking antibody in the complex comprises CDRs set forth in SEQ ID NOs: 12-17. In some embodiments, the non-blocking antibody in the complex comprises a light chain variable region set for in the SEQ ID NO: 19. In some embodiments, the non-blocking antibody in the complex comprises a heavy chain variable region set for in the SEQ ID NO: 18.

In some embodiments, the soluble MIC in the complex is sMICA. In some embodiments, the sMICA comprises an amino acid sequence set forth one of SEQ ID NOs: 1 and 20-77. In some embodiments, the soluble MIC in the complex is sMICB. In some embodiments, the sMICB comprises an amino acid sequence set forth one of SEQ ID NOs: 2 and 78-100.

Compositions comprising the NKG2D complex described herein and a pharmaceutically acceptable carrier, diluent or adjuvant are also contemplated.

In another aspect, described herein is a method of activating CD8 T cells in a subject in need thereof, comprising administering to the subject a complex comprising a soluble MHC I chain-related molecule (sMIC) and a non-blocking sMIC-neutralizing antibody.

In some embodiments, the subject is suffering from a viral infection. In some embodiments, the viral infection is caused by a DNA Virus (e.g., Herpes Viruses such as Herpes Simplex virus, Epstein-Barr virus, Cytomegalovirus; Pox viruses such as Variola (small pox) virus; Hepadnaviruses (e.g, Hepatitis B virus); Papilloma viruses; Adenovinises); RNA Viruses (e.g., HIV I, II; HTLV I, II; Poliovirus; Hepatitis A; coronoviruses, such as sudden acute respiratory syndrome (SARS); Orthomyxoviruses (e.g., Influenza viruses); Paramyxoviruses (e.g., Measles virus); Rabies virus; Hepatitis C virus), Flaviviruses, Influenza viruses; caliciviruses; or rabies viruses, rinderpest viruses and Arena virus. In some embodiments, the viral infection is caused by Lymphocytic choriomeningitis (LCMV).

In some embodiments, the subject is suffering from cancer. Exemplary cancers include, but are not limited to, basal cell carcinoma, biliary tract cancer, bladder cancer, bone cancer, brain and CNS cancer, breast cancer, cancer of the peritoneum, cervical cancer, choriocarcinoma, colon and rectum cancer, connective tissue cancer, cancer of the digestive system, endometrial cancer, esophageal cancer, eye cancer, cancer of the head and neck, gastric cancer, gastrointestinal cancer, glioblastoma (GBM), hepatic carcinoma, hepatoma, intra-epithelial neoplasm, renal cancer, larynx cancer, leukemia, liver cancer, lung cancer, small-cell lung cancer, non-small cell lung cancer, adenocarcinoma of the lung, squamous carcinoma of the lung, lymphoma including Hodgkin's and non-Hodgkin's lymphoma, melanoma, myeloma, neuroblastoma, oral cavity cancer (e.g., lip, tongue, mouth, and pharynx), ovarian cancer, pancreatic cancer, prostate cancer, retinoblastoma, rhabdomyosarcoma, rectal cancer, cancer of the respiratory system, salivary gland carcinoma, sarcoma, skin cancer, squamous cell cancer, stomach cancer, testicular cancer, thyroid cancer, uterine or endometrial cancer, cancer of the urinary system, vulval cancer, B-cell lymphoma (including low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, mantle cell lymphoma, AIDS-related lymphoma, Waldenstrom's Macroglobulinemia, chronic lymphocytic leukemia (CLL), acute lymphoblastic leukemia (ALL), Hairy cell leukemia, chronic myeloblastic leukemia, and post-transplant lymphoproliferative disorder (PTLD).

In some embodiments, the subject is suffering from a MHC I chain-related molecule (MIC)-negative cancer. In some embodiments, the subject is suffering from a viral infection.

In some embodiments, the methods described herein further comprises administering an immune checkpoint inhibitor to the subject. In some embodiments, the immune checkpoint inhibitor is MGA27, ipilimumab, pembrolizumab, nivolumab, atezolizumab, IMP321, IPH2101, tremelimumab, pidilizumab, MPDL3280A, MEDI4736, MSB0010718C, AUNP12, avelumab, durvalumab or TSR-022.

### BRIEF DESCRIPTION OF THE FIGURES

FIG. 1 demonstrates that the sMIC/anti-MIC D4H3 mAb complex provides strong co-stimulation to amplify CD3-mediated CD8 T cell activation.

FIG. 2 shows that sMIC/NO4 mAb complex co-stimulation amplifies antigen-specific TCR-signaling. Human tyrosinase-specific HLA-A2-restrcited TIL13831 was co-cultured overnight with APC T2-A2 cells under indicated condition before functional assay.

FIG. 3 shows that the co-stimulation with the sMIC/D4H3 mAb complex amplifies antigen-specific TCR-signaling. Human tyrosinase-specific HLA-A2-restrcited TIL13831 was co-cultured o/n with APC T2-A2 cells under indicated condition before functional assay.

FIG. 4 is a graph showing that therapy with sMIC/D4H3 inhibited MIC colon tumor growth. 4 mg/Kg BW of all reagents (control IgG, antibody D4H3 and sMIC/D4H3 complex) were administrated i.p. twice weekly. *, P<0.01.

FIGS. 5A and 5B provide graphs showing the detection of sMIC(A/B)/D4H3 complex or sMIC(A/B)/NO4 complex binding to NKG2D. Recombinant soluble NKG2D-His tagged (2 ug/ml) was immobilized to the 96-well plate overnight at 4C. 50 μl of recombinant sMICA or sMICB as indicated concentration was mixed with various amount of D4H3 (FIG. 5A) or NO4 (FIG. 5B). The concentration of D4H3 or NO4 is indicated on the X-axis. After incubation and washes, binding to NKG2D by the SMIC/D4H3 or sMIC/NO4 complex was detected by HRP-conjugated goat anti-mouse IgG.

FIG. 6 depicts MICA alleles set forth in SEQ ID NOs: 20-77.

FIG. 7 depicts MICB alleles set forth in SEQ ID NOs 78-100.

FIG. 8 is a graph showing that the administration of the sMIC/D4H3 complex to mice inolulated with lympcytic choriomeningitis virus (LCMV) significantly reduced LCMV titer in the mice.

### DETAILED DESCRIPTION

Described herein is a Natural Killer Group 2D (NKG2D) complex a soluble MHC I chain-related molecule (sMIC) and a non-blocking sMIC-neutralizing antibody. Also described herein is a fusion protein comprising sMIC linked to a heavy chain (or a light chain) of the non-blocking sMIC-neutralizing antibody with a polylinker. As shown in the Examples provided herein: 1) in contrast to soluble NKG2D ligands, sMIC/anti-sMIC complex provides a durable magnitude of NKG2D co-stimulation to amplify TCR/CD3 signaling; 2) sMIC/anti-sMIC complex and CD28 agonist produce additive co-stimulatory effect; 3) in contrast to negative effect of soluble NKG2D ligands that downmodulate NKG2D expression, sMIC/anti-sMIC co-stimulation stabilizes NKG2D expression on CD8 T cells.

### Major Histocompatibility Complex Class I Chain-Related (MIC) Polypeptides

The NKG2D superagonist complex (or fusion protein) described herein comprises a Major Histocompatibility Complex class I chain-related (MIC) polypeptide. MICs are surface transmembrane proteins. The presence of a MIC polypeptide on the cell surface can signal the immune receptor NKG2D for tumor immune destruction, typically by natural killer cells (NK cells) and cytotoxic T cells (CTLs). However, in many tumors, MIC is shed from the tumor surface, resulting in decreased host immunity against the tumor cell and promoting tumor evasion and progression. MIC polypeptides include, but are not limited to the human MICA (e.g. NCBI Ref Seqs NP_000238 (SEQ ID NO:1) and 001170990) and human MICB (e.g. NCBI Ref Seq: NP 005922 (SEQ ID NO: 2). In some embodiments, the MIC polypeptide comprises MICA. In some embodiments, the MIC polypeptide can comprise MICB. In some embodiments, the MIC polypeptide comprises the following amino acid sequence:

(SEQ ID NO: 3)
EPHSLRYNLTVLSWDGSVQSGFLAEVHLDGQPFLRYDRQKCRAKPQG
QWAEDVLGNKTWDRETRDLTGNGKDLRMTLAHIKDQKEGLHSLQEIR
VCEIHEDNSTRSSQHFYYDGELFLSQNVETEEWTVPQSSRAQTLAMN
VRNFLKEDAMKTKTHYHAMHADCLQELRRYLESSVVLRRRVPPMVNV
TRSEALEGNITVTCGASSFYPRNITLTWRQDGVSLSHDTQQWGDVLP
DGNGTYQTWVATRICQGEEQRFTCYMEHSGNHSTHPVPS.

In some embodiments, the NKG2D complex (or fusion protein) comprises a soluble MIC (sMIC) polypeptide. As used herein “soluble MIC” or “sMIC” refers to a portion of a MIC polypeptide that is lacking a transmembrane domain, e.g., an extracellular portion of MIC that has been cleaved from the transmembrane domain. In some embodiments, soluble MIC can comprise about, e.g., amino acids 24-260 of SEQ ID NOs: 1 or 2. In some embodiments, soluble MIC can comprise about, e.g., 20 or more amino acids of residues 24-260 of SEQ ID NO: 1 or 2, e.g., 20, 50, 100, 150 or more amino acids of residues 24-260 of SEQ ID NO: 1 or 2.

In some embodiments, the NK2GD agonist complex (or fusion protein) comprises an MICA allele set forth in one of SEQ ID NOs: 20-77. In some embodiments, the NK2GD agonist complex (or fusion protein) comprises an MICB allele set forth in one of SEQ ID NOs: 78-100.

### Anti-MIC Antibodies

In some embodiments, the NKG2D agonist complex (or fusion protein) described herein comprises a non-blocking antibody or antigen-binding portion thereof which selectively binds to a MIC polypeptide. In some embodiments, the MIC polypeptide is a soluble MIC polypeptide (sMIC).

The term “non-blocking antibody” as used herein refers to a sMIC-neutralizing antibody that does not block the interaction of NKG2D with membrane-bound MIC or soluble MIC and thus does not interfere with sensitivity of NKG2D-mediated NK cell cytolytic activity against MIC+ cells.

As used herein, the term “antibody” refers to immunoglobulin molecules. The term also refers to antibodies comprised of two immunoglobulin heavy chains and two immunoglobulin light chains as well as a variety of forms including full length antibodies and antigen-binding portions thereof; including, for example, an immunoglobulin molecule, a monoclonal antibody, a chimeric antibody, a CDR-grafted antibody, a humanized antibody, a a single domain antibody (dAb), a diabody, a multispecific antibody, a dual specific antibody, an anti-idiotypic antibody, a bispecific antibody, a functionally active epitope-binding fragment thereof, bifunctional hybrid antibodies (e.g., Lanzavecchia et al., Eur. J. Immunol 17, 105 (1987)) and single chains (e.g., Huston et al., Proc. Natl. Acad. Sci. U.S.A., 85, 5879-5883 (1988) and Bird et al., Science 242, 423-426 (1988), which are incorporated herein by reference). (See, generally, Hood et al, Immunology, Benjamin, N.Y., 2ND ed. (1984), Harlow and Lane, Antibodies. A Laboratory Manual, Cold Spring Harbor Laboratory (1988) and Hunkapiller and Hood, Nature, 323, 15-16 (1986), which are incorporated herein by reference).

Each heavy chain is composed of a variable region of said heavy chain (abbreviated here as HCVR or VH) and a constant region of said heavy chain. The heavy chain constant region consists of three domains CH1, CH2 and CH3. Each light chain is composed of a variable region of said light chain (abbreviated here as LCVR or VL) and a constant region of said light chain. The light chain constant region consists of a CL domain. The VH and VL regions may be further divided into hypervariable regions referred to as complementarity-determining regions (CDRs) and interspersed with conserved regions referred to as framework regions (FR). Each VH and VL region thus consists of three CDRs and four FRs which are arranged from the N terminus to the C terminus in the following order: FR1, CDR1, FR2, CDR2, FR3, CDR3, FR4. This structure is well known to those skilled in the art.

As used herein, the term “CDR” refers to the complementarity determining regions within antibody variable sequences. There are three CDRs in each of the variable regions of the heavy chain and of the light chain, which are designated CDR1, CDR2 and CDR3, for each of the variable regions. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Other boundaries defining CDRs overlapping with the Kabat CDRs have been described by Padlan (FASEB J. 9:133-139 (1995)) and MacCallum (J Mol Biol 262(5):732-45 (1996)) and Chothia (J. Mol. Biol. 196:901-917 (1987) and Nature 342:877-883 (1989)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat defined CDRs.

As used herein, “selectively binds” or “specifically binds” refers to the ability of an anti-MIC-binding peptide (e.g., an antibody or portion thereof) described herein to bind to a target, such as a MIC molecule present on the cell-surface, with a KD of 10⁻⁵M (10000 nM) or less, e.g., 10⁻⁶ M, 10⁻⁷M, 10⁻⁸M, 10⁻⁹ M, 10⁻¹⁰ M, 10¹¹ M, 10⁻¹² M, or less (or any range comprising any of these values as endpoints). Specific binding can be influenced by, for example, the affinity and avidity of the polypeptide agent and the concentration of polypeptide agent. The person of ordinary skill in the art can determine appropriate conditions under which the polypeptide agents described herein selectively bind the targets using any suitable methods, such as titration of a polypeptide agent in a suitable cell binding assay. A polypeptide specifically bound to a target is not displaced by a non-similar competitor. In certain embodiments, an antibody or antigen-binding portion thereof is said to specifically bind an antigen when it preferentially recognizes its target antigen in a complex mixture of proteins and/or macromolecules. In some embodiments, the antibody or antigen-binding portion thereof binds to a sMIC polypeptide with a dissociation constant (KD) of 10⁻⁵ M (10000 nM) or less, e.g., 10⁻⁵ M, 10⁻⁶ M, 10⁻⁷ M, 10⁻⁸ M, 10⁻⁹ M, 10¹⁰ M, 10¹¹ M, 10⁻¹² M, or less.

The terms “antigen-binding fragment” or “antigen-binding portion” of an antibody, used interchangeably herein, refer to one or more fragments of an antibody as described herein which still demonstrate the binding affinities as defined above herein. Fragments of a complete antibody have been shown to be able to carry out the antigen-binding function of an antibody. Examples of antigen-binding fragments include, but are not limited to, (i) an Fab fragment, i.e. a monovalent fragment composed of the VL, VH, CL and CH1 domains; (ii) an F(ab′)2 fragment, i.e., a bivalent fragment comprising two Fab fragments linked to one another in the hinge region via a disulfide bridge; (iii) an Fd fragment composed of the VH and CH1 domains; (iv) an Fv fragment composed of the FL and VH domains of a single arm of an antibody; and (v) a dAb fragment (Ward et al., (1989) Nature 341:544-546) consisting of a VH domain or of VH, CH1, CH2, DH3, or VH, CH2, CH3 (dAbs, or single domain antibodies, comprising only VL domains have also been shown to specifically bind to target epitopes). Although the two domains of the Fv fragment, namely VL and VH, are encoded by separate genes, they may further be linked to one another using a synthetic linker, e.g., a poly-G4S amino acid sequence, and recombinant methods, making it possible to prepare them as a single protein chain in which the VL and VH regions combine in order to form monovalent molecules (known as single chain Fv (ScFv); see, for example, Bird et al. (1988) Science 242:423-426; and Huston et al. (1988) Proc. Natl. Acad. Sci. USA 85:5879-5883). The term “antigen-binding portion” of an antibody is also intended to comprise such single chain antibodies. Other forms of single chain antibodies such as “diabodies” are likewise included here. Diabodies are bivalent, bispecific antibodies in which VH and VL domains are expressed on a single polypeptide chain, but using a linker which is too short for the two domains being able to combine on the same chain, thereby forcing said domains to pair with complementary domains of a different chain and to form two antigen-binding sites (see, for example, Holliger, R, et al. (1993) Proc. Natl. Acad. Sci. USA 90:64446448; Poljak, R. J, et al. (1994) Structure 2:1121-1123). An immunoglobulin constant domain refers to a heavy or light chain constant domain. Human IgG heavy chain and light chain constant domain amino acid sequences are known in the art.

Furthermore, an antibody as described herein or an antigen-binding portion thereof may be part of a larger immunoadhesion molecule formed by covalent or noncovalent association of said antibody or antibody portion with one or more further proteins or peptides. Relevant to such immunoadhesion molecules are the use of the streptavidin core region in order to prepare a tetrameric scFv molecule (Kipriyanov, S. M., et al. (1995) Human Antibodies and Hybridomas 6:93-101) and the use of a cystein residue, a marker peptide and a C-terminal polyhistidinyl, e.g. hexahistidinyl tag (‘hexahistidinyl tag’ disclosed as SEQ ID NO: 18) in order to produce bivalent and biotinylated scFv molecules (Kipriyanov, S. M., et al. (1994) Mol. Immunol. 31:10471058).

In some embodiments, the antibody is an IgG, a monoclonal antibody, a chimeric antibody, a CDR-grafted antibody, a humanized antibody, a multispecific antibody, a dual specific antibody, an anti-idiotypic antibody or a bispecific antibody. In some embodiments, the antigen binding portion of the antibody is a Fab, a Fab′, a F(ab′)2, a Fv, a disulfide linked Fv, a scFv, a single domain antibody, a diabody or a functionally active epitope-binding fragment thereof.

The term “human antibody” refers to antibodies whose variable and constant regions correspond to or are derived from immunoglobulin sequences of the human germ line, as described, for example, by Kabat et al. (see Kabat, et al. (1991) Sequences of Proteins of Immunological Interest, Fifth Edition, U.S. Department of Health and Human Services, NIH Publication No. 91-3242). However, the human antibodies can contain amino acid residues not encoded by human germ line immunoglobulin sequences (for example mutations which have been introduced by random or site-specific mutagenesis in vitro or by somatic mutation in vivo), for example in the CDRs, and in particular in CDR3. Recombinant human antibodies as described herein have variable regions and may also contain constant regions derived from immunoglobulin sequences of the human germ line (see Kabat, E. A., et al. (1991) Sequences of Proteins of Immunological Interest, Fifth Edition, U.S. Department of Health and Human Services, NIH Publication No. 91-3242). According to particular embodiments, however, such recombinant human antibodies are subjected to in vitro mutagenesis (or to a somatic in-vivo mutagenesis, if an animal is used which is transgenic due to human Ig sequences) so that the amino acid sequences of the VH and VL regions of the recombinant antibodies are sequences which although related to or derived from VH and VL sequences of the human germ line, do not naturally exist in vivo within the human antibody germ line repertoire. According to particular embodiments, recombinant antibodies of this kind are the result of selective mutagenesis or back mutation or of both. Preferably, mutagenesis leads to an affinity to the target which is greater, and/or an affinity to non-target structures which is smaller than that of the parent antibody.

The term “chimeric antibody” refers to antibodies which contain sequences for the variable region of the heavy and light chains from one species and constant region sequences from another species, such as antibodies having murine heavy and light chain variable regions linked to human constant regions. Humanized antibodies have variable region framework residues substantially from a human antibody (termed an acceptor antibody) and complementarity determining regions substantially from a non-human antibody, e.g. a mouse-antibody, (referred to as the donor immunoglobulin). See, Queen et al., Proc Natl Acad Sci USA 86:10029-10033 (1989) and WO 90/07861, U.S. Pat. Nos. 5,693,762, 5,693,761, 5,585,089, 5,530,101 and Winter, U.S. Pat. No. 5,225,539, which are herein incorporated by reference in their entirety. The constant region(s), if present, are also substantially or entirely from a human immunoglobulin. The human variable domains are usually chosen from human antibodies whose framework sequences exhibit a high degree of sequence identity with the (murine) variable region domains from which the CDRs were derived. The heavy and light chain variable region framework residues can be substantially similar to a region of the same or different human antibody sequences. The human antibody sequences can be the sequences of naturally occurring human antibodies or can be consensus sequences of several human antibodies. See Carter et al., WO 92/22653, which is herein incorporated by reference in its entirety.

In some embodiments, the antibodies described herein are not naturally-occurring biomolecules. For example, a murine antibody raised against an antigen of human origin would not occur in nature absent human intervention and manipulation, e.g., manufacturing steps carried out by a human. Chimeric antibodies are also not naturally-occurring biomolecules, e.g., in that they comprise sequences obtained from multiple species and assembled into a recombinant molecule. In certain particular embodiments, the human antibody reagents described herein are not naturally-occurring biomolecules, e.g., fully human antibodies directed against a human antigen would be subject to negative selection in nature and are not naturally found in the human body.

One of ordinary skill in the art will recognize that individual substitutions, deletions or additions to an amino acid sequence which alters a single amino acid or a small percentage of amino acids in the encoded sequence is a “conservatively modified variant” where the alteration results in the substitution of an amino acid with a chemically similar amino acid and retain the ability to specifically bind the target antigen (e.g., an epitope present on sMIC) of a MIC polypeptide). Such conservatively modified variants are in addition to and do not exclude polymorphic variants, interspecies homologs, and alleles consistent with the disclosure.

In some embodiments, the antibody or antigen-binding portion thereof specifically binds a sMIC polypeptide and comprises one or more heavy and light chain complementarity determining regions (CDRs) selected from the group consisting of: (a) a light chain CDR1 having the amino acid sequence of SEQ ID NO: 4; (b) a light chain CDR2 having the amino acid sequence of SEQ ID NO: 5; (c) a light chain CDR3 having the amino acid sequence of SEQ ID NO: 6; (d) a heavy chain CDR1 having the amino acid sequence of SEQ ID NO: 7; (e) a heavy chain CDR2 having the amino acid sequence of SEQ ID NO: 8; and (f) a heavy chain CDR3 having the amino acid sequence of SEQ ID NO: 9. In some embodiments, the antibody or antigen-binding fragment thereof described herein comprises one or more CDRs, e.g. 1 CDR, 2 CDRs, 3 CDRs, 4 CDRs, 5 CDRs, or 6 CDRs, selected from the group consisting of (a) a light chain CDR1 having the amino acid sequence of SEQ ID NO: 4; (b) a light chain CDR2 having the amino acid sequence of SEQ ID NO: 5; (c) a light chain CDR3 having the amino acid sequence of SEQ ID NO: 6; (d) a heavy chain CDR1 having the amino acid sequence of SEQ ID NO: 7; (e) a heavy chain CDR2 having the amino acid sequence of SEQ ID NO: 8; and (f) a heavy chain CDR3 having the amino acid sequence of SEQ ID NO: 9. In some embodiments, the antibody or antigen-binding fragment thereof comprises a heavy chain or a portion thereof, comprising one or more CDRs, e.g., 1 CDR, 2 CDRs, or 3 CDRs selected from the group consisting of a heavy chain CDR1 having the amino acid sequence of SEQ ID NO: 7; a heavy chain CDR2 having the amino acid sequence of SEQ ID NO: 8; and a heavy chain CDR3 having the amino acid sequence of SEQ ID NO: 9. In some embodiments, the antibody or antigen-binding fragment thereof comprises a light chain or a portion thereof, comprising one or more CDRs, e.g., 1 CDR, 2 CDRs, or 3 CDRs selected from the group consisting of a light chain CDR1 having the amino acid sequence of SEQ ID NO: 4; a light chain CDR2 having the amino acid sequence of SEQ ID NO: 5; a light chain CDR3 having the amino acid sequence of SEQ ID NO: 6.

In some embodiments, the antibody or antigen-binding portion thereof comprises light chain complementarity determining regions (CDRs): (a) a light chain CDR1 having the amino acid sequence of SEQ ID NO: 12; (b) a light chain CDR2 having the amino acid sequence of SEQ ID NO: 13; and (c) a light chain CDR3 having the amino acid sequence of SEQ ID NO: 14. In some embodiments, the antibody or antigen-binding portion comprises the heavy chain complementarity determining regions (CDRs): (d) a heavy chain CDR1 having the amino acid sequence of SEQ ID NO: 15; (e) a heavy chain CDR2 having the amino acid sequence of SEQ ID NO: 16; and (f) a heavy chain CDR3 having the amino acid sequence of SEQ ID NO: 17. In some embodiments, the antibody or antigen-binding fragment thereof described herein comprises one or more CDRs, e.g. 1 CDR, 2 CDRs, 3 CDRs, 4 CDRs, 5 CDRs, or 6 CDRs, selected from the group consisting of (a) a light chain CDR1 having the amino acid sequence of SEQ ID NO: 12; (b) a light chain CDR2 having the amino acid sequence of SEQ ID NO: 13; (c) a light chain CDR3 having the amino acid sequence of SEQ ID NO: 14; (d) a heavy chain CDR1 having the amino acid sequence of SEQ ID NO: 15; (e) a heavy chain CDR2 having the amino acid sequence of SEQ ID NO: 16; and (f) a heavy chain CDR3 having the amino acid sequence of SEQ ID NO: 17. In some embodiments, the antibody or antigen-binding fragment thereof comprises a heavy chain or a portion thereof, comprising one or more CDRs, e.g., 1 CDR, 2 CDRs, or 3 CDRs selected from the group consisting of a heavy chain CDR1 having the amino acid sequence of SEQ ID NO: 15; a heavy chain CDR2 having the amino acid sequence of SEQ ID NO: 16; and a heavy chain CDR3 having the amino acid sequence of SEQ ID NO: 17. In some embodiments, the antibody or antigen-binding fragment thereof comprises a light chain or a portion thereof, comprising one or more CDRs, e.g., 1 CDR, 2 CDRs, or 3 CDRs selected from the group consisting of a light chain CDR1 having the amino acid sequence of SEQ ID NO: 13; a light chain CDR2 having the amino acid sequence of SEQ ID NO: 12; a light chain CDR3 having the amino acid sequence of SEQ ID NO: 14.

In some embodiments, the antibody or antigen-binding fragment thereof comprises a heavy chain variable region comprising the amino acid sequence of SEQ ID NO: 11. In some embodiments, the antibody or antigen-binding fragment thereof comprises a light chain variable region comprising the amino acid sequence of SEQ ID NO: 10. In some embodiments, the antibody or antigen-binding fragment thereof comprises a heavy chain comprising the amino acid sequence of SEQ ID NO: 11. In some embodiments, the antibody or antigen-binding fragment thereof comprises a light chain comprising the amino acid sequence of SEQ ID NO: 10.

In some embodiments, the antibody or antigen-binding fragment thereof comprises a heavy chain variable region comprising the amino acid sequence of SEQ ID NO: 19. In some embodiments, the antibody or antigen-binding fragment thereof comprises a light chain variable region comprising the amino acid sequence of SEQ ID NO: 18. In some embodiments, the antibody or antigen-binding fragment thereof comprises a heavy chain comprising the amino acid sequence of SEQ ID NO: 19. In some embodiments, the antibody or antigen-binding fragment thereof comprises a light chain comprising the amino acid sequence of SEQ ID NO: 18.

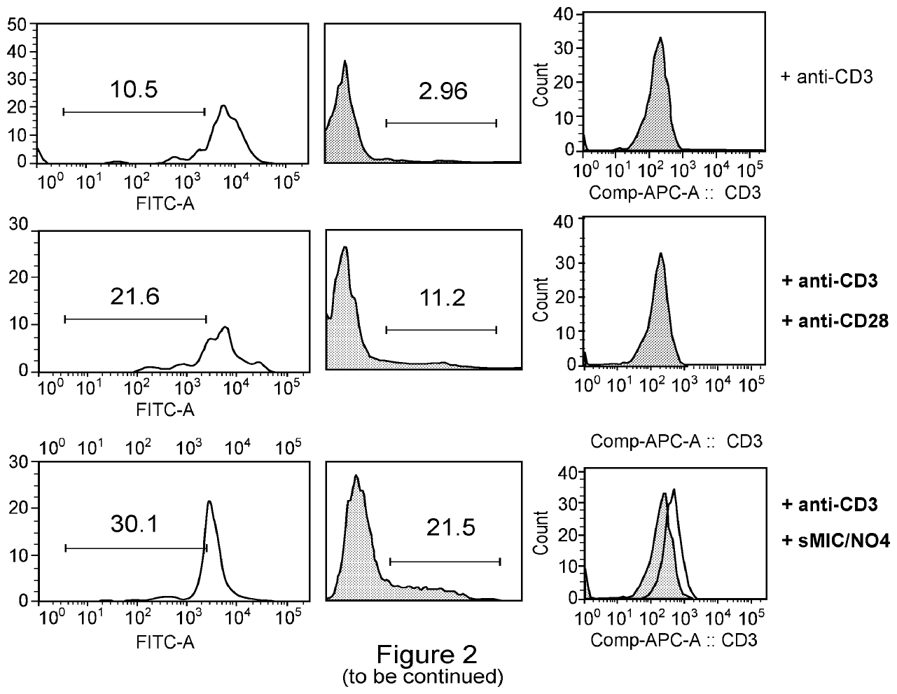

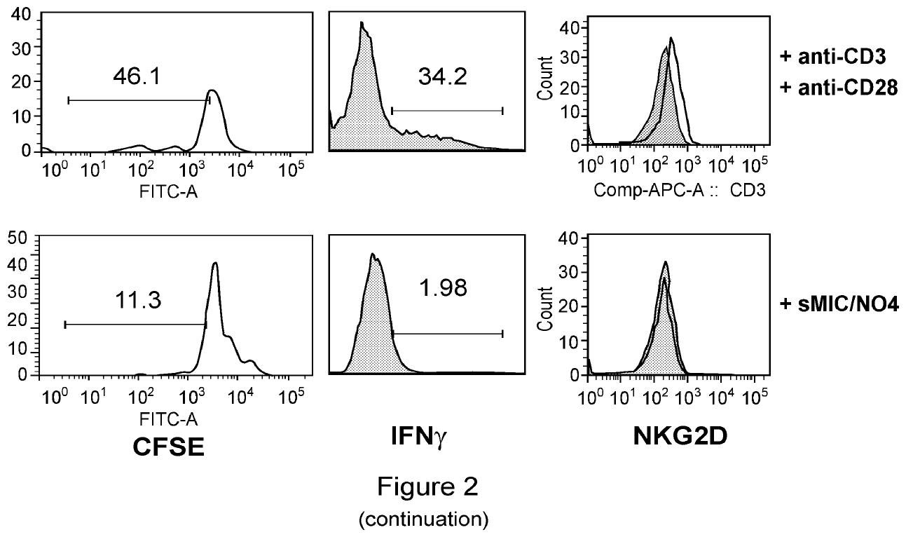

In embodiments wherein an antibody as described herein comprises at least one CDR which is not identical to the sequence of SEQ ID NOs: 4-9 or 12-17, the amino acid sequence of that at least one CDR can be selected by methods well known to one of skill in the art. For example, Fujii, 2004, “Antibody affinity maturation by random mutagenesis” in Methods in Molecular Biology: Antibody Engineering 248: 345-349 (incorporated by reference herein in its entirety), particularly at FIG. 2 and Section 3.3, describes methods of generating a library for any CDR of interest. This allows one of ordinary skill in the art to identify alternative CDRs, including conservative substitution variants of the specific CDR sequences described herein, which, when present in an antibody or antigen-binding fragment thereof as described herein, will result in an antigen or antigen-binding fragment thereof which will bind a MIC polypeptide, but will not block the MIC polypeptide from binding to other antibodies. The method described in Fujii et al. also permits one of ordinary skill in the art to screen for a light chain sequence which will give the desired binding behavior when combined with a known heavy chain fragment and vice versa.

In some embodiments, the antibody and/or antigen-binding portion thereof described herein can be a variant of a sequence described herein, e.g., a conservative substitution variant of an antibody polypeptide. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity, e.g. antigen-specific binding activity for the relevant target polypeptide, e.g., a sMIC polypeptide. A wide variety of PCR-based site-specific mutagenesis approaches are also known in the art and can be applied by the ordinarily skilled artisan.

Examples of substitution variants include conservative substitution of amino acids, e.g., in a V_{H} or V_{L}, domain, that do not alter the sequence of a CDR. A conservative substitution in a sequence not comprised by a CDR can be a substitution relative to a wild-type or naturally-occurring sequence, e.g., human or murine framework and/or constant regions of an antibody sequence.

In some embodiments, a conservatively modified variant of an antibody can comprise alterations other than in the CDRs, e.g. a conservatively modified variant of an antibody reagent can comprise CDRs having the sequence of one or more of SEQ ID NOs: 4-9 and 12-17.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g. antigen-binding activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H).

Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class.

Particular conservative substitutions include, for example; Ala into Gly or into Ser; Arg into Lys; Asn into Gln or into H is; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments, the antibody comprises an amino acid sequence at least 90%, at least 91%, at least 92%, at least 93%, at least 94%, at least 95%, at least 96%, at least 97%, at least 98%, at least 99%, or more, identical to any of the CDR sequences set forth in SEQ ID NOs: 4-9 and 12-17 or any of the variable region amino acid sequence set forth in SEQ ID NOs: 10, 11, 18 and 19. The degree of homology (percent identity) between a native and a mutant sequence can be determined, for example, by comparing the two sequences using freely available computer programs commonly employed for this purpose on the world wide web (e.g., BLASTp or BLASTn with default settings).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties.

Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

### Methods of Making Antibodies

Traditionally, monoclonal antibodies have been produced as native molecules in murine hybridoma lines. In addition to that technology, the methods and compositions described herein provide for recombinant DNA expression of monoclonal antibodies. This allows the production of humanized antibodies as well as a spectrum of antibody derivatives and complexes in a host species of choice. The production of antibodies in bacteria, yeast, transgenic animals and chicken eggs are also alternatives for hybridoma-based production systems. The main advantages of transgenic animals are potential high yields from renewable sources.

Nucleic acid molecules encoding amino acid sequence variants of antibodies are prepared by a variety of methods known in the art. These methods include, but are not limited to preparation by oligonucleotide-mediated (or site-directed) mutagenesis, PCR mutagenesis, and cassette mutagenesis of an earlier prepared variant or a non-variant version of the antibody. A nucleic acid sequence encoding at least one antibody, portion or polypeptide as described herein can be recombined with vector DNA in accordance with conventional techniques, including blunt-ended or staggered-ended termini for ligation, restriction enzyme digestion to provide appropriate termini, filling in of cohesive ends as appropriate, alkaline phosphatase treatment to avoid undesirable joining, and ligation with appropriate ligases. Techniques for such manipulations are disclosed, e.g., by Maniatis et al., Molecular Cloning, Lab. Manual (Cold Spring Harbor Lab. Press, N Y, 1982 and 1989), and Ausubel, 1987, 1993, and can be used to construct nucleic acid sequences which encode a monoclonal antibody or antigen binding region thereof.

In some embodiments, the introduced nucleotide sequence is incorporated into a plasmid or viral vector capable of autonomous replication in the recipient host. Any of a wide variety of vectors can be employed for this purpose and are known and available to those or ordinary skill in the art. See, e.g., Ausubel et al., 1987, 1993. Factors of importance in selecting a particular plasmid or viral vector include: the ease with which recipient cells that contain the vector may be recognized and selected from those recipient cells which do not contain the vector; the number of copies of the vector which are desired in a particular host; and whether it is desirable to be able to “shuttle” the vector between host cells of different species.

Example prokaryotic vectors known in the art include plasmids such as those capable of replication in E. coli., for example. Other gene expression elements useful for the expression of cDNA encoding antibodies or antigen-binding portions thereof include, but are not limited to (a) viral transcription promoters and their enhancer elements, such as the SV40 early promoter. (Okayama et al., 3 Mol. Cell. Biol. 280 (1983)), Rous sarcoma virus LTR (Gorman et al., 79 PNAS 6777 (1982)), and Moloney murine leukemia virus LTR (Grosschedl et al., 41 Cell 885 (1985)); (b) splice regions and polyadenylation sites such as those derived from the SV40 late region (Okayarea et al., 1983), and (c) polyadenylation sites such as in SV40 (Okayama et al., 1983) Immunoglobulin cDNA genes can be expressed as described by Liu et al., infra, and Weidle et al., 51 Gene 21 (1987), using as expression elements the SV40 early promoter and its enhancer, the mouse immunoglobulin H chain promoter enhancers, SV40 late region mRNA splicing, rabbit S-globin intervening sequence, immunoglobulin and rabbit S-globin polyadenylation sites, and SV40 polyadenylation elements.

Each fused gene is assembled in, or inserted into, an expression vector. Recipient cells capable of expressing the chimeric immunoglobulin chain gene product are then transfected singly with an antibody, antigen-binding portion thereof, or chimeric H or chimeric L chain-encoding gene, or are co-transfected with a chimeric H and a chimeric L chain gene. The transfected recipient cells are cultured under conditions that permit expression of the incorporated genes and the expressed immunoglobulin chains or intact antibodies or fragments are recovered from the culture.

An expression vector carrying an antibody, or antigen-binding portion thereof as described herein can be introduced into an appropriate host cell by any of a variety of suitable means, including such biochemical means as transformation, transfection, conjugation, protoplast fusion, calcium phosphate-precipitation, and application with polycations such as diethylaminoethyl (DEAE) dextran, and such mechanical means as electroporation, direct microinjection, and microprojectile bombardment. Johnston et al., 240 Science 1538 (1988), as known to one of ordinary skill in the art.

Host mammalian cells can be grown in vitro or in vivo. Mammalian cells provide post-translational modifications to immunoglobulin protein molecules including leader peptide removal, folding and assembly of H and L chains, glycosylation of the antibody molecules, and secretion of functional antibody protein.

Mammalian cells which can be useful as hosts for the production of antibody proteins, in addition to the cells of lymphoid origin described above, include cells of fibroblast origin, such as Vero (ATCC CRL 81) or CHO-K1 (ATCC CRL 61) cells. Exemplary eukaryotic cells that can be used to express polypeptides include, but are not limited to, COS cells, including COS 7 cells; 293 cells, including 293-6E cells; CHO cells, including CHO—S and DG44 cells; PER.C6™ cells (Crucell); and NSO cells. In some embodiments, a particular eukaryotic host cell is selected based on its ability to make desired post-translational modifications to the heavy chains and/or light chains. For example, in some embodiments, CHO cells produce polypeptides that have a higher level of sialylation than the same polypeptide produced in 293 cells.

In some embodiments, one or more antibodies or antigen-binding portions thereof as described herein can be produced in vivo in an animal that has been engineered or transfected with one or more nucleic acid molecules encoding the polypeptides, according to any suitable method.

In some embodiments, an antibody or antigen-binding portion thereof as described herein is produced in a cell-free system. Nonlimiting exemplary cell-free systems are described, e.g., in Sitaraman et al., Methods Mol. Biol. 498: 229-44 (2009); Spirin, Trends Biotechnol. 22: 538-45 (2004); Endo et al., Biotechnol. Adv. 21: 695-713 (2003), the disclosure of which are incorporated herein by reference in their entirety.

In some aspects, provided herein are methods and systems for the production of a humanized antibody, which is prepared by a process which comprises maintaining a host transformed with a first expression vector which encodes the light chain of the humanized antibody and with a second expression vector which encodes the heavy chain of the humanized antibody under such conditions that each chain is expressed and isolating the humanized antibody formed by assembly of the thus-expressed chains. The first and second expression vectors can be the same vector. Also provided herein are DNA sequences encoding the light chain or the heavy chain of the humanized antibody; an expression vector which incorporates a said DNA sequence; and a host transformed with a said expression vector.

Generating a humanized antibody from the sequences and information provided herein can be practiced by those of ordinary skill in the art without undue experimentation. In one approach, there are four general steps employed to humanize a monoclonal antibody, see, e.g., U.S. Pat. Nos. 5,585,089; 6,835,823; 6,824,989. These are: (1) determining the nucleotide and predicted amino acid sequence of the starting antibody light and heavy variable domains; (2) designing the humanized antibody, i.e., deciding which antibody framework region to use during the humanizing process; (3) the actual humanizing methodologies/techniques; and (4) the transfection and expression of the humanized antibody.

Usually the CDR regions in humanized antibodies and human antibody variants are substantially identical, and more usually, identical to the corresponding CDR regions in the mouse or human antibody from which they were derived. Although not usually desirable, it is sometimes possible to make one or more conservative amino acid substitutions of CDR residues without appreciably affecting the binding affinity of the resulting humanized immunoglobulin or human antibody variant. Occasionally, substitutions of CDR regions can enhance binding affinity.

In addition, techniques developed for the production of “chimeric antibodies” (see Morrison et al., Proc. Natl. Acad. Sci. 81:851-855 (1984); Neuberger et al., Nature 312:604-608 (1984); Takeda et al., Nature 314:452-454 (1985); which are incorporated by reference herein in their entireties) by splicing genes from a mouse, or other species, antibody molecule of appropriate antigen specificity together with genes from a human antibody molecule of appropriate biological activity can be used. A chimeric antibody is a molecule in which different portions are derived from different animal species, such as those having a variable region derived from a murine monoclonal antibody and a human immunoglobulin constant region, e.g., humanized antibodies.

The variable segments of chimeric antibodies are typically linked to at least a portion of an immunoglobulin constant region (Fc), typically that of a human immunoglobulin. Human constant region DNA sequences can be isolated in accordance with well-known procedures from a variety of human cells, such as immortalized B-cells (WO 87/02671; which is incorporated by reference herein in its entirety). The antibody can contain both light chain and heavy chain constant regions. The heavy chain constant region can include CH1, hinge, CH2, CH3, and, sometimes, CH4 regions. For therapeutic purposes, the CH2 domain can be deleted or omitted.

Alternatively, techniques described for the production of single chain antibodies (see, e.g. U.S. Pat. No. 4,946,778; Bird, Science 242:423-42 (1988); Huston et al., Proc. Natl. Acad. Sci. USA 85:5879-5883 (1988); and Ward et al., Nature 334:544-54 (1989); which are incorporated by reference herein in their entireties) can be adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli can also be used (see, e.g. Skerra et al., Science 242:1038-1041 (1988); which is incorporated by reference herein in its entirety).

### Methods of Treatment

In one aspect, described herein is a method of activating CD8 T cells in a subject in need thereof, comprising administering to the subject a complex (or fusion protein) comprising a soluble MHC I chain-related molecule (sMIC) and a non-blocking sMIC-neutralizing antibody.

In some embodiments, the subject is suffering from a viral infection. There are many viruses for which CD8+ T cells have been shown to play a role in protection. See Huber et al., Front. Immunol., 5:171, 2014. In some embodiments, the subject is suffering from a viral infection caused by a DNA Virus (e.g., Herpes Viruses such as Herpes Simplex virus, Epstein-Barr virus, Cytomegalovirus; Pox viruses such as Variola (small pox) virus; Hepadnaviruses (e.g, Hepatitis B virus); Papilloma viruses; Adenovinises); RNA Viruses (e.g., HIV I, II; HTLV I, II; Poliovirus; Hepatitis A; coronoviruses, such as sudden acute respiratory syndrome (SARS); Orthomyxoviruses (e.g., Influenza viruses); Paramyxoviruses (e.g., Measles virus); Rabies virus; Hepatitis C virus), Flaviviruses, Influenza viruses; caliciviruses; rabies viruses, rinderpest viruses, Arena virus, and the like. In some embodiments, the viral infection is caused by Lymphocytic choriomeningitis (LCMV). In some embodiments, the subject is suffering from a virus-related disease. Exemplary virus-related diseases include, but are not limited to, Acquired immunodeficiency; Hepatitis; Gastroenteritis; Hemorrhagic diseases; Enteritis; Carditis; Encephalitis; Paralysis; Bronchiolitis; Upper and lower respiratory disease; Respiratory Papillomatosis; Arthritis; Disseminated disease, Meningitis and Mononucleosis.

In some embodiments, the subject is suffering from a cancer or a malignancy. In some embodiments, the activation of CD8 T cells is determined by enzyme-linked immunospot (ELISPOT), Flowcytometry activated sorting (FACS) or a target killing cytotoxicity assay. Other methods of determining activation of T cells, such as those described in Plebanski et al., Expert. Rev. Vaccines, 9:595-600, 2010.

A “tumor” as used herein refers to an uncontrolled growth of cells which interferes with the normal functioning of the bodily organs and systems. A subject that has a cancer or a tumor is a subject having objectively measurable cancer cells present in the subject's body. Included in this definition are benign tumors and malignant cancers, as well as potentially dormant tumors or micrometastases. Cancers which migrate from their original location and seed other vital organs can eventually lead to the death of the subject through the functional deterioration of the affected organs. Hematopoietic cancers, such as leukemia, are able to out-compete the normal hematopoietic compartments in a subject, thereby leading to hematopoietic failure (in the form of anemia, thrombocytopenia and neutropenia) ultimately causing death.

Exemplary cancers include, but are not limited to, carcinoma, lymphoma, blastoma, sarcoma, and leukemia. More particular examples of such cancers include, but are not limited to, basal cell carcinoma, biliary tract cancer; bladder cancer; bone cancer; brain and CNS cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma (GBM); hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); lymphoma including Hodgkin's and non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; as well as other carcinomas and sarcomas; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD).

In some embodiments, the tumor or malignancy is MIC-negative. As used herein, the term “MIC-negative tumor” is used to describe a tumor cell, a cluster of tumor cells or a tumor mass, which does not produce a MIC protein. This term is intended to encompass all tumor cells and/or tumor masses that do not display a MIC protein on the tumor cell surface, thus these cells do not shed MIC protein. In other words, subjects suffering from a MIC-negative cancer should have no detectable sMIC beyond background noise. MIC-negative tumors can be identified by assaying serum levels MIC (e.g., sMICA or sMICb) using standard MICA or MICB detection ELISA as described in, for example, Ghadially et al., Br. J. Cancer, 116:1208-1217, 2017, the disclosure of which is incorporated herein by reference. For tumors that a biopsy is available, tumors that are negative for MIC expression can also be selected or confirmed by immunohistochemistry showing no cross-reactivity with an anti-MIC antibody.

As used herein, a “subject” means a human or animal. In some embodiments, the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments, the subject is a mammal, e.g., a primate, e.g., a human. The terms, “patient”, “individual” and “subject” are used interchangeably herein.

In some embodiments, the subject is a mammal. The mammal can be a human, non-human primate, mouse, rat, dog, cat, horse, or cow, but are not limited to these examples. Mammals other than humans can be advantageously used, for example, as subjects that represent animal models of, for example, various cancers. In addition, the methods described herein can be used to treat domesticated animals and/or pets. A subject can be male or female.

In some embodiments, the subject has been previously diagnosed with or identified as suffering from or having a condition in need of treatment (e.g., a cancer) or one or more complications related to such a condition, and optionally, but need not have already undergone treatment for a condition or the one or more complications related to the condition. Alternatively, the subject has not been previously diagnosed as having a condition in need of treatment or one or more complications related to such a condition. For example, a subject can be one who exhibits one or more risk factors for a condition or one or more complications related to a condition or a subject who does not exhibit risk factors. A “subject in need” of treatment for a particular condition can be a subject having that condition, diagnosed as having that condition, or at risk of developing that condition.

As used herein, the terms “treat,” “treatment,” “treating,” or “amelioration” when used in reference to a disease, disorder or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, alleviation of one or more symptom(s), diminishment of extent of the deficit, stabilized (i.e., not worsening) state of a tumor or malignancy, delay or slowing of tumor growth and/or metastasis, and an increased lifespan as compared to that expected in the absence of treatment.

As used herein, the term “administering,” refers to the placement of the agonist complex (or fusion protein) described herein into a subject by a method or route which results in at least partial localization of the agents at a desired site. The pharmaceutical composition comprising an the agonist complex (or fusion protein) described herein can be administered by any appropriate route which results in an effective treatment in the subject.

Pharmaceutical Compositions and Routes of Administration

Compositions comprising a Natural Killer Group 2D (NKG2D) agonist complex (or fusion protein) comprising a soluble MHC I chain-related molecule (sMIC) and a non-blocking sMIC-neutralizing antibody as described herein are also contemplated. In some embodiments, the composition is a pharmaceutical composition. As used herein, the term “pharmaceutical composition” refers to the active agent in combination with a carrier accepted for use in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The preparation of a pharmaceutical composition that contains active ingredients dissolved or dispersed therein is well understood in the art and need not be limited based on formulation. Typically such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents, pH buffering agents and the like which enhance or maintain the effectiveness of the active ingredient. The therapeutic composition as described herein can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used in the invention that will be effective in the treatment of a particular disorder or condition will depend on the nature of the disorder or condition, and can be determined by standard clinical techniques.

Therapeutic compositions containing at least one agent can be conventionally administered in a unit dose, for example. The term “unit dose” when used in reference to a therapeutic composition refers to physically discrete units suitable as unitary dosage for the subject, each unit containing a predetermined quantity of active material calculated to produce the desired therapeutic effect in association with the required physiologically acceptable diluent, i.e., carrier, or vehicle.

Precise amounts of active ingredient required to be administered depend on the judgment of the practitioner and are particular to each individual. However, suitable dosage ranges for systemic application are disclosed herein and depend on the route of administration. Suitable regimes for administration are also variable, but are typified by an initial administration followed by repeated doses at one or more hour intervals by a subsequent injection or other administration. Alternatively, continuous intravenous infusion sufficient to maintain concentrations in the blood in the ranges specified for in vivo therapies are contemplated.

As used herein, the phrase “therapeutically effective amount”, “effective amount” or “effective dose” refers to an amount that provides a therapeutic or aesthetic benefit in the treatment, prevention, or management of a tumor or malignancy, e.g., an amount that provides a statistically significant decrease in at least one symptom, sign, or marker of a tumor or malignancy. Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

The dosage ranges for the agent depend upon the potency, and encompass amounts large enough to produce the desired effect e.g., slowing of tumor growth or a reduction in tumor size. The dosage should not be so large as to cause unacceptable adverse side effects. Generally, the dosage will vary with the age, condition, and sex of the patient and can be determined by one of skill in the art. The dosage can also be adjusted by the individual physician in the event of any complication. In some embodiments, the dosage ranges from 0.001 mg/kg body weight to 0.5 mg/kg body weight. Alternatively, the dose range can be titrated to maintain serum levels between 1 mg/mL and 1000 mg/mL. For systemic administration, subjects can be administered a therapeutic amount, such as, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

Administration of the doses recited above can be repeated. In some embodiments, the doses are given once a day, or multiple times a day. In some embodiments, the doses are administered daily for several weeks or months. The duration of treatment depends upon the subject's clinical progress and responsiveness to therapy.

In some embodiments, the dose is from about 2 mg/kg to about 15 mg/kg. In some embodiments, the dose is about 2 mg/kg. In some embodiments, the dose is about 4 mg/kg. In some embodiments, the dose is about 5 mg/kg. In some embodiments, the dose is about 6 mg/kg. In some embodiments, the dose is about 8 mg/kg. In some embodiments, the dose is about 10 mg/kg. In some embodiments, the dose is about 15 mg/kg.

In some embodiments, the dose can be administered intravenously. In some embodiments, the intravenous administration can be an infusion occurring over a period of from about 10 minute to about 3 hours. In some embodiments, the intravenous administration can be an infusion occurring over a period of from about 30 minutes to about 90 minutes.

In some embodiments the dose can be administered about weekly. In some embodiments, the dose can be administered weekly. In some embodiments, the dose can be administered weekly for from about 12 weeks to about 18 weeks. In some embodiments the dose can be administered about every 2 weeks. In some embodiments the dose can be administered about every 3 weeks. In some embodiments, the dose can be from about 2 mg/kg to about 15 mg/kg administered about every 2 weeks. In some embodiments, the dose can be from about 2 mg/kg to about 15 mg/kg administered about every 3 weeks. In some embodiments, the dose can be from about 2 mg/kg to about 15 mg/kg administered intravenously about every 2 weeks. In some embodiments, the dose can be from about 2 mg/kg to about 15 mg/kg administered intravenously about every 3 weeks.

In some embodiments, the dose can be from about 1 mg to about 2000 mg. In some embodiments, the dose can be about 3 mg. In some embodiments, the dose can be about 10 mg. In some embodiments, the dose can be about 30 mg. In some embodiments, the dose can be about 1000 mg. In some embodiments, the dose can be about 2000 mg. In some embodiments, the dose can be about 3 mg given by intravenous infusion daily. In some embodiments, the dose can be about 10 mg given by intravenous infusion daily. In some embodiments, the dose can be about 30 mg given by intravenous infusion three times per week.

A therapeutically effective amount is an amount of an agent that is sufficient to produce a statistically significant, measurable change in tumor size, tumor growth etc. (efficacy measurements are described below herein). Such effective amounts can be gauged in clinical trials as well as animal studies.

An agent can be administered intravenously by injection or by gradual infusion over time. Given an appropriate formulation for a given route, for example, agents useful in the methods and compositions described herein can be administered intravenously, intranasally, by inhalation, intraperitoneally, intramuscularly, subcutaneously, intracavity, and can be delivered by peristaltic means, if desired, or by other means known by those skilled in the art. It is preferred that the compounds used herein are administered orally, intravenously or intramuscularly to a patient having cancer. Local administration directly to a tumor mass is also specifically contemplated.

Combination Therapies

Combination of the NKG2D agonist complex (or fusion protein) described with an additional therapeutic agent or therapy is specifically contemplated. In some embodiments, the additional therapeutic agent is effective in the treatment of cancer. Exemplary additional therapeutics or therapies include, but are not limited to, a surgical therapy, chemotherapy (e.g., administration of a protein kinase inhibitor or a EGFR-targeted therapy), radiation therapy, cryotherapy, hyperthermia treatment, phototherapy, radioablation therapy, hormonal therapy, immunotherapy, small molecule therapy, receptor kinase inhibitor therapy, anti-angiogenic therapy, cytokine therapy or a biological therapies such as monoclonal antibodies, siRNA, miRNA, antisense oligonucleotides, ribozymes or gene therapy. Without limitation the biological therapy may be a gene therapy, such as tumor suppressor gene therapy, a cell death protein gene therapy, a cell cycle regulator gene therapy, a cytokine gene therapy, a toxin gene therapy, an immunogene therapy, a suicide gene therapy, a prodrug gene therapy, an anti-cellular proliferation gene therapy, an enzyme gene therapy, or an anti-angiogenic factor gene therapy.

The combination therapy may precede or follow the other agent treatment by intervals ranging from minutes to weeks. In embodiments where the other agent and combination therapy are applied separately to the cell, one would generally ensure that a significant period of time did not expire between the time of each delivery, such that the agent and expression construct would still be able to exert an advantageously combined effect on the cell. In such instances, it is contemplated that one may contact the cell with both modalities within about 12-24 hours of each other and, more preferably, within about 6-12 hours of each other. In some situations, it may be desirable to extend the time period for treatment significantly, however, where several days (e.g., 2, 3, 4, 5, 6 or 7) to several weeks (e.g., 1, 2, 3, 4, 5, 6, 7 or 8) lapse between the respective administrations.

In some embodiments, the additional therapeutic or therapy comprises chemotherapy. Exemplary chemotherapies include, but are not limited to, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, famesyl-protein transferase inhibitors, transplatinum, 5-fluorouracil, vincristine, vinblastine and methotrexate, Temazolomide (an aqueous form of DTIC), alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids such as retinoic acid; capecitabine; carboplatin, procarbazine, plicomycin, gemcitabien, navelbine, farnesyl-protein transferase inhibitors, transplatinum; and pharmaceutically acceptable salts, acids or derivatives of any of the above.

In some embodiments, the agonist complexes described herein are used in combination with histone deacetylase inhibitors. In some embodiments, the agonist complexes described herein are used in combination with gefitinib. In some embodiments, the agonist complexes described herein are used in combination with Gleevec (e.g., from about 400 to about 800 mg/day of Gleevec may be administered to a patient). In some embodiments, one or more chemotherapeutic agents may be used in combination with the agonist complexes described herein.

In some embodiments, the additional therapeutic or therapy comprises radiotherapy. Other factors that cause DNA damage and have been used extensively include what are commonly known as y-rays, X-rays, and/or the directed delivery of radioisotopes to tumor cells. Other forms of DNA damaging factors are also known such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

In some embodiments, the additional therapeutic of therapy comprises immunotherapy. Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells as well as genetically engineered variants of these cell types modified to express chimeric antigen receptors.

Exemplary immunotherapies that can be combined with the agonist complexes described herein include immune adjuvants (e.g., Mycobacterium bovis, Plasmodium falciparum, dinitrochlorobenzene and aromatic compounds) (U.S. Pat. Nos. 5,801,005; 5,739,169; Hui and Hashimoto, 1998; Christodoulides et al., 1998), cytokine therapy (e.g., interferons .alpha., .beta. and .gamma.; interleukins (IL-1, IL-2), GM-CSF and TNF) (Bukowski et al., 1998; Davidson et al., 1998; Hellstrand et al., 1998) gene therapy (e.g., TNF, IL-1, IL-2, p53) (Qin et al., 1998; Austin-Ward and Villaseca, 1998; U.S. Pat. Nos. 5,830,880 and 5,846,945) and monoclonal antibodies (e.g., anti-ganglioside GM2, anti-HER-2, anti-p185) (Pietras et al., 1998; Hanibuchi et al., 1998; U.S. Pat. No. 5,824,311). Herceptin (trastuzumab) is a chimeric (mouse-human) monoclonal antibody that blocks the HER2-neu receptor. It possesses anti-tumor activity and has been approved for use in the treatment of malignant tumors (Dillman, 1999). Combination therapy of cancer with herceptin and chemotherapy has been shown to be more effective than the individual therapies. Thus, it is contemplated that one or more anti-cancer therapies may be employed with the combination therapy described herein.

Other immunotherapies contemplated for use in methods of the present disclosure include those described by Tchekmedyian et al., 2015, incorporated herein by reference. The immunotherapy may comprise suppression of T regulatory cells (Tregs), myeloid derived suppressor cells (MDSCs) and cancer associated fibroblasts (CAFs). In some embodiments, the immunotherapy is a tumor vaccine (e.g., whole tumor cell vaccines, peptides, and recombinant tumor associated antigen vaccines), or adoptive cellular therapies (ACT) (e.g., T cells, natural killer cells, TILs, and LAK cells). The T cells may be engineered with chimeric antigen receptors (CARs) or T cell receptors (TCRs) to specific tumor antigens. As used herein, a chimeric antigen receptor (or CAR) may refer to any engineered receptor specific for an antigen of interest that, when expressed in a T cell, confers the specificity of the CAR onto the T cell. Once created using standard molecular techniques, a T cell expressing a chimeric antigen receptor may be introduced into a patient, as with a technique such as adoptive cell transfer. In some aspects, the T cells are activated CD4 and/or CD8 T cells in the individual which are characterized by IFNγ “producing CD4 and/or CD8 T cells and/or enhanced cytolytic activity relative to prior to the administration of the combination. The CD4 and/or CD8 T cells may exhibit increased release of cytokines selected from the group consisting of IFN-γ, TNF-α and interleukins. The CD4 and/or CD8 T cells can be effector memory T cells. In certain embodiments, the CD4 and/or CD8 effector memory T cells are characterized by having the expression of CD44^{high} CD62^{low}.

Examples of monoclonal antibodies that may be used in combination with the compositions provided herein include, without limitation, trastuzumab (anti-HER2/neu antibody); Pertuzumab (anti-HER2 mAb); cetuximab (chimeric monoclonal antibody to epidermal growth factor receptor EGFR); panitumumab (anti-EGFR antibody); nimotuzumab (anti-EGFR antibody); Zalutumumab (anti-EGFR mAb); Necitumumab (anti-EGFR mAb); MDX-210 (humanized anti-HER-2 bispecific antibody); MDX-210 (humanized anti-HER-2 bispecific antibody); MDX-447 (humanized anti-EGF receptor bispecific antibody); Rituximab (chimeric murine/human anti-CD20 mAb); Obinutuzumab (anti-CD20 mAb); Ofatumumab (anti-CD20 mAb); Tositumumab-I131 (anti-CD20 mAb); Ibritumomab tiuxetan (anti-CD20 mAb); Bevacizumab (anti-VEGF mAb); Ramucirumab (anti-VEGFR2 mAb); Ranibizumab (anti-VEGF mAb); Aflibercept (extracellular domains of VEGFR1 and VEGFR2 fused to IgG1 Fc); AMG386 (angiopoietin-1 and -2 binding peptide fused to IgG1 Fc); Dalotuzumab (anti-IGF-1R mAb); Gemtuzumab ozogamicin (anti-CD33 mAb); Alemtuzumab (anti-Campath-1/CD52 mAb); Brentuximab vedotin (anti-CD30 mAb); Catumaxomab (bispecific mAb that targets epithelial cell adhesion molecule and CD3); Naptumomab (anti-5T4 mAb); Girentuximab (anti-Carbonic anhydrase ix); or Farletuzumab (anti-folate receptor). Other examples include antibodies such as Panorex™ (17-1A) (murine monoclonal antibody); Panorex (@ (17-1A) (chimeric murine monoclonal antibody); BEC2 (ami-idiotypic mAb, mimics the GD epitope) (with BCG); Oncolym (Lym-1 monoclonal antibody); SMART M195 Ab, humanized 13′ 1 LYM-1 (Oncolym), Ovarex (B43.13, anti-idiotypic mouse mAb); 3622W94 mAb that binds to EGP40 (17-1A) pancarcinoma antigen on adenocarcinomas; Zenapax (SMART Anti-Tac (IL-2 receptor); SMART M195 Ab, humanized Ab, humanized); NovoMAb-G2 (pancarcinoma specific Ab); TNT (chimeric mAb to histone antigens); TNT (chimeric mAb to histone antigens); Gliomab-H (Monoclonals-Humanized Abs); GNI-250 Mab; EMD-72000 (chimeric-EGF antagonist); LymphoCide (humanized IL.L.2 antibody); and MDX-260 bispecific, targets GD-2, ANA Ab, SMART IDIO Ab, SMART ABL 364 Ab or ImmuRAIT-CEA. Examples of antibodies include those disclosed in U.S. Pat. Nos. 5,736,167, 7,060,808, and 5,821,337.

Further examples of antibodies include anti-human OX40 agonist antibody (Genentech); Zanulimumab (anti-CD4 mAb), Keliximab (anti-CD4 mAb); Ipilimumab (MDX-101; anti-CTLA-4 mAb); Tremilimumab (anti-CTLA-4 mAb); (Daclizumab (anti-CD25/IL-2R mAb); Basiliximab (anti-CD25/IL-2R mAb); MDX-1106 (anti-PD1 mAb); antibody to GITR; GC1008 (anti-TGF-.beta. antibody); metelimumab/CAT-192 (anti-TGF-.beta. antibody); lerdelimumab/CAT-152 (anti-TGF-.beta. antibody); ID11 (anti-TGF-.beta. antibody); Denosumab (anti-RANKL mAb); BMS-663513 (humanized anti-4-1BB mAb); SGN-40 (humanized anti-CD40 mAb); CP870,893 (human anti-CD40 mAb); Infliximab (chimeric anti-TNF mAb; Adalimumab (human anti-TNF mAb); Certolizumab (humanized Fab anti-TNF); Golimumab (anti-TNF); Etanercept (Extracellular domain of TNFR fused to IgG1 Fc); Belatacept (Extracellular domain of CTLA-4 fused to Fc); Abatacept (Extracellular domain of CTLA-4 fused to Fc); Belimumab (anti-B Lymphocyte stimulator); Muromonab-CD3 (anti-CD3 mAb); Otelixizumab (anti-CD3 mAb); Teplizumab (anti-CD3 mAb); Tocilizumab (anti-IL6R mAb); REGN88 (anti-IL6R mAb); Ustekinumab (anti-IL-12/23 mAb); Briakinumab (anti-IL-12/23 mAb); Natalizumab (anti-.alpha.4 integrin); Vedolizumab (anti-.alpha.4.beta.7 integrin mAb); T1 h (anti-CD6 mAb); Epratuzumab (anti-CD22 mAb); Efalizumab (anti-CD11a mAb); and Atacicept (extracellular domain of transmembrane activator and calcium-modulating ligand interactor fused with Fc).

It is contemplated that other agents may be used in combination with the compositions provided herein to improve the therapeutic efficacy of treatment. These additional agents include immunomodulatory agents, agents that affect the upregulation of cell surface receptors and GAP junctions, cytostatic and differentiation agents, inhibitors of cell adhesion, or agents that increase the sensitivity of the hyperproliferative cells to apoptotic inducers. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL would potentiate the apoptotic inducing abilities of the compositions provided herein by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the compositions provided herein to improve the anti-hyerproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the compositions provided herein to improve the treatment efficacy.

In further embodiments, the other agents may be one or more oncolytic viruses. Examples of oncolytic viruses include adenoviruses, adeno-associated viruses, retroviruses, lentiviruses, herpes viruses, pox viruses, vaccinia viruses, vesicular stomatitis viruses, polio viruses, Newcastle's Disease viruses, Epstein-Barr viruses, influenza viruses and reoviruses. In a particular embodiment, the other agent is talimogene laherparepvec (T-VEC) which is an oncolytic herpes simplex virus genetically engineered to express GM-CSF. Talimogene laherparepvec, HSV-1 [strain JS1] ICP34.5-/ICP47-/hGM-CSF, (previously known as OncoVEX.sup.GM CSF) is an intratumorally delivered oncolytic immunotherapy comprising an immune-enhanced HSV-1 that selectively replicates in solid tumors. (Lui et al., 2003; U.S. Pat. Nos. 7,223,593 and 7,537,924; incorporated herein by reference).

In certain embodiments, hormonal therapy may also be used in conjunction with the present embodiments or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

In some aspects, the additional anti-cancer agent is a protein kinase inhibitor or a monoclonal antibody that inhibits receptors involved in protein kinase or growth factor signaling pathways such as an EGFR, VEGFR, AKT, Erb1, Erb2, ErbB, Syk, Bcr-Abl, JAK, Src, GSK-3, PI3K, Ras, Raf, MAPK, MAPKK, mTOR, c-Kit, eph receptor or BRAF inhibitors. Nonlimiting examples of protein kinase or growth factor signaling pathways inhibitors include Afatinib, Axitinib, Bevacizumab, Bosutinib, Cetuximab, Crizotinib, Dasatinib, Erlotinib, Fostamatinib, Gefitinib, Imatinib, Lapatinib, Lenvatinib, Mubritinib, Nilotinib, Panitumumab, Pazopanib, Pegaptanib, Ranibizumab, Ruxolitinib, Saracatinib, Sorafenib, Sunitinib, Trastuzumab, Vandetanib, AP23451, Vemurafenib, MK-2206, GSK690693, A-443654, VQD-002, Miltefosine, Perifosine, CAL101, PX-866, LY294002, rapamycin, temsirolimus, everolimus, ridaforolimus, Alvocidib, Genistein, Selumetinib, AZD-6244, Vatalanib, P1446A-05, AG-024322, ZD1839, P276-00, GW572016 or a mixture thereof.

In some aspects, the PI3K inhibitor is selected from the group of PI3K inhibitors consisting of buparlisib, idelalisib, BYL-719, dactolisib, PF-05212384, pictilisib, copanlisib, copanlisib dihydrochloride, ZSTK-474, GSK-2636771, duvelisib, GS-9820, PF-04691502, SAR-245408, SAR-245409, sonolisib, Archexin, GDC-0032, GDC-0980, apitolisib, pilaralisib, DLBS 1425, PX-866, voxtalisib, AZD-8186, BGT-226, DS-7423, GDC-0084, GSK-21 26458, INK-1 1 17, SAR-260301, SF-1 1 26, AMG-319, BAY-1082439, CH-51 32799, GSK-2269557, P-7170, PWT-33597, CAL-263, RG-7603, LY-3023414, RP-5264, RV-1729, taselisib, TGR-1 202, GSK-418, INCB-040093, Panulisib, GSK-105961 5, CNX-1351, AMG-51 1, PQR-309, 17beta-Hydroxywortmannin, AEZS-129, AEZS-136, HM-5016699, IPI-443, ONC-201, PF-4989216, RP-6503, SF-2626, X-339, XL-499, PQR-401, AEZS-132, CZC-24832, KAR-4141, PQR-31 1, PQR-316, RP-5090, VS-5584, X-480, AEZS-126, AS-604850, BAG-956, CAL-130, CZC-24758, ETP-46321, ETP-471 87, GNE-317, GS-548202, HM-032, KAR-1 139, LY-294002, PF-04979064, PI-620, PKI-402, PWT-143, RP-6530, 3-HOI-BA-01, AEZS-134, AS-041 164, AS-252424, AS-605240, AS-605858, AS-606839, BCCA-621 C, CAY-10505, CH-5033855, CH-51 08134, CUDC-908, CZC-1 9945, D-106669, D-87503, DPT-NX7, ETP-46444, ETP-46992, GE-21, GNE-123, GNE-151, GNE-293, GNE-380, GNE-390, GNE-477, GNE-490, GNE-493, GNE-614, HMPL-51 8, HS-104, HS-106, HS-1 16, HS-173, HS-196, IC-486068, INK-055, KAR 1 141, KY-1 2420, Wortmannin, Lin-05, NPT-520-34, PF-04691503, PF-06465603, PGNX-01, PGNX-02, PI 620, PI-103, PI-509, PI-516, PI-540, PIK-75, PWT-458, RO-2492, RP-5152, RP-5237, SB-201 5, SB-2312, SB-2343, SHBM-1009, SN 32976, SR-13179, SRX-2523, SRX-2558, SRX-2626, SRX-3636, SRX-5000, TGR-5237, TGX-221, UCB-5857, WAY-266175, WAY-266176, EI-201, AEZS-131, AQX-MN100, KCC-TGX, OXY-1 1 1 A, PI-708, PX-2000, and WJD-008.

It is contemplated that the additional cancer therapy can comprise an antibody, peptide, polypeptide, small molecule inhibitor, siRNA, miRNA or gene therapy which targets, for example, epidermal growth factor receptor (EGFR, EGFR1, ErbB-1, HER1), ErbB-2 (HER2/neu), ErbB-3/HER3, ErbB-4/HER4, EGFR ligand family; insulin-like growth factor receptor (IGFR) family, IGF-binding proteins (IGFBPs), IGFR ligand family (IGF-1R); platelet derived growth factor receptor (PDGFR) family, PDGFR ligand family; fibroblast growth factor receptor (FGFR) family, FGFR ligand family, vascular endothelial growth factor receptor (VEGFR) family, VEGF family; HGF receptor family: TRK receptor family; ephrin (EPH) receptor family; AXL receptor family; leukocyte tyrosine kinase (LTK) receptor family; TIE receptor family, angiopoietin 1, 2; receptor tyrosine kinase-like orphan receptor (ROR) receptor family; discoidin domain receptor (DDR) family; RET receptor family; KLG receptor family; RYK receptor family; MuSK receptor family; Transforming growth factor alpha (TGF-α), TGF-α receptor; Transforming growth factor-beta (TGF-.beta.), TGF-.beta. receptor; Interleukin 13 receptor alpha2 chain (1L13Ralpha2), Interleukin-6 (IL-6), 1L-6 receptor, Interleukin-4, IL-4 receptor, Cytokine receptors, Class I (hematopoietin family) and Class II (interferon/1L-10 family) receptors, tumor necrosis factor (TNF) family, TNF-α, tumor necrosis factor (TNF) receptor superfamily (TNTRSF), death receptor family, TRAIL-receptor; cancer-testis (CT) antigens, lineage-specific antigens, differentiation antigens, alpha-actinin-4, ARTC1, breakpoint cluster region-Abelson (Bcr-abl) fusion products, B-RAF, caspase-5 (CASP-5), caspase-8 (CASP-8), beta-catenin (CTNNB1), cell division cycle 27 (CDC27), cyclin-dependent kinase 4 (CDK4), CDKN2A, COA-1, dek-can fusion protein, EFTUD-2, Elongation factor 2 (ELF2), Ets variant gene 6/acute myeloid leukemia 1 gene ETS (ETC6-AML1) fusion protein, fibronectin (FN), GPNMB, low density lipid receptor/GDP-L fucose: beta-Dgalactose 2-alpha-Lfucosyltraosferase (LDLR/FUT) fusion protein, HLA-A2, arginine to isoleucine exchange at residue 170 of the alpha-helix of the alpha2-domain in the HLA-A2 gene (HLA-A*201-R1700, MLA-A11, heat shock protein 70-2 mutated (HSP70-2M), KIAA0205, MART2, melanoma ubiquitous mutated 1, 2, 3 (MUM-1, 2, 3), prostatic acid phosphatase (PAP), neo-PAP, Myosin class 1, NFYC, OGT, OS-9, pml-RARalpha fusion protein, PRDXS, PTPRK, K-ras (KRAS2), N-ras (NRAS), HRAS, RBAF600, SIRT2, SNRPD1, SYT-SSX1 or -SSX2 fusion protein, Triosephosphate Isomerase, BAGE, BAGE-1, BAGE-2,3,4,5, GAGE-1,2,3,4,5,6,7,8, GnT-V (aberrant N-acetyl giucosaminyl transferase V, MGATS), HERV-K-MEL, KK-LC, LAGE, LAGE-1, CTL-recognized antigen on melanoma (CAMEL), MAGE-A1 (MAGE-1), MAGE-A2, MAGE-A3, MAGE-A4, MAGE-AS, MAGE-A6, MAGE-A8, MAGE-A9, MAGE-A10, MAGE-A11, MAGE-A12, MAGE-3, MAGE-B1, MAGE-B2, MAGE-B5, MAGE-B6, MAGE-C1, MAGE-C2, mucin 1 (MUC1), MART-1/Melan-A (MLANA), gp100, gp100/Pme117 (S1LV), tyrosinase (TYR), TRP-1, HAGE, NA-88, NY-ESO-1, NY-ESO-1/LAGE-2, SAGE, Sp17, SSX-1,2,3,4, TRP2-1NT2, carcino-embryonic antigen (CEA), Kallikfein 4, mammaglobm-A, OA1, prostate specific antigen (PSA), prostate specific membrane antigen, TRP-1/gp75, TRP-2, adipophilin, interferon inducible protein absent in nielanorna 2 (AIM-2), BING-4, CPSF, cyclin D1, epithelial cell adhesion molecule (Ep-CAM), EpbA3, fibroblast growth factor-5 (FGF-5), glycoprotein 250 (gp250intestinal carboxyl esterase (iCE), alpha-feto protein (AFP), M-CSF, mdm-2, MUC1, p53 (TP53), PBF, FRAME, PSMA, RAGE-1, RNF43, RU2AS, SOX10, STEAP1, survivin (BIRCS), human telomerase reverse transcriptase (hTERT), telomerase, Wilms' tumor gene (WT1), SYCP1, BRDT, SPANX, XAGE, ADAM2, PAGE-5, LIP1, CTAGE-1, CSAGE, MMA1, CAGE, BORIS, HOM-TES-85, AF15q14, HCA66I, LDHC, MORC, SGY-1, SPO11, TPX1, NY-SAR-35, FTHLI7, NXF2 TDRD1, TEX 15, FATE, TPTE, immunoglobulin idiotypes, Bence-Jones protein, estrogen receptors (ER), androgen receptors (AR), CD40, CD30, CD20, CD19, CD33, CD4, CD25, CD3, cancer antigen 72-4 (CA 72-4), cancer antigen 15-3 (CA 15-3), cancer antigen 27-29 (CA 27-29), cancer antigen 125 (CA 125), cancer antigen 19-9 (CA 19-9), beta-human chorionic gonadotropin, 1-2 microglobulin, squamous cell carcinoma antigen, neuron-specific enoJase, heat shock protein gp96, GM2, sargramostim, CTLA-4, 707 alanine proline (707-AP), adenocarcinoma antigen recognized by T cells 4 (ART-4), carcinoembryogenic antigen peptide-1 (CAP-1), calcium-activated chloride channel-2 (CLCA2), cyclophilin B (Cyp-B), human signet ring tumor-2 (HST-2), Human papilloma virus (HPV) proteins (HPV-E6, HPV-E7, major or minor capsid antigens, others), Epstein-Barr vims (EBV) proteins (EBV latent membrane proteins-LMP1, LMP2; others), Hepatitis B or C virus proteins, and HIV proteins.

In some embodiments, the methods described herein comprise administering a checkpoint inhibitor to the subject. In some embodiments, the checkpoint inhibitor is a small molecule, an inhibitory nucleic acid, an inhibitory polypeptide, antibody or antigen-binding domain thereof, or antibody reagent. In some embodiments, the checkpoint inhibitor is an antibody or antigen-binding domain thereof, or antibody reagent binds an immune checkpoint polypeptide and inhibits its activity. Common checkpoints that are targeted for therapeutics include, but are not limited to PD-L1, PD-L2, PD-1, CTLA-4, TIM-3, LAG-3, VISTA, and TIGIT. In some embodiments, the checkpoint inhibitor is an antibody or antigen-binding domain thereof, or antibody reagent binds a PD-1, PD-L1, or PD-L2 polypeptide and inhibits its activity.

Inhibitors of known checkpoint regulators (e.g., PD-L1, PD-L2, PD-1, CTLA-4, TIM-3, LAG-3, VISTA, or TIGIT) are known in the art. Non-limiting examples of checkpoint inhibitors (with checkpoint targets and manufacturers noted in parentheses) can include: MGA271 (B7-H3: MacroGenics); ipilimumab (CTLA-4; Bristol Meyers Squibb); pembrolizumab (PD-1; Merck); nivolumab (PD-1; Bristol Meyers Squibb); atezolizumab (PD-L1; Genentech); IMP321 (LAG3: Immuntep); BMS-986016 (LAG3; Bristol Meyers Squibb); IPH2101 (KIR; Innate Pharma); tremelimumab (CTLA-4; Medimmune); pidilizumab (PD-1; Medivation); MPDL3280A (PD-L1; Roche); MEDI4736 (PD-L1; Astra7eneca); MSB0010718C (PD-L1; EMD Serono); AUNP12 (PD-1; Aurigene); avelumab (PD-L1; Merck); durvalumab (PD-L1; Medimmune); and TSR-022 (TIM3; Tesaro).

In some embodiments, the checkpoint inhibitor inhibits PD-1. PD-1 inhibitors include, but are not limited to Pembrolizumab (Keytruda™), Nivolumab, AUNP-12, and Pidilizumab. In another embodiment, the checkpoint inhibitor inhibits PD-L1. PD-L1 inhibitors include, but are not limited to Atezolizumab, MPDL3280A, Avelumab, and Durvalumab.

### Monitoring Efficacy of Treatment

The efficacy of a given treatment for cancer can be determined by the skilled clinician. However, a treatment is considered “effective treatment,” as the term is used herein, if any one or all of the signs or symptoms of e.g., a tumor are altered in a beneficial manner or other clinically accepted symptoms are improved, or even ameliorated, e.g., by at least 10% following treatment with an agent as described herein. Efficacy can also be measured by a failure of an individual to worsen as assessed by hospitalization or need for medical interventions (i.e., progression of the disease is halted). Methods of measuring these indicators are known to those of skill in the art and/or described herein.

An effective amount for the treatment of a disease means that amount which, when administered to a mammal in need thereof, is sufficient to result in effective treatment as that term is defined herein, for that disease. Efficacy of an agent can be determined by assessing physical indicators of, for example cancer, e.g., tumor size, tumor mass, tumor density, angiogenesis, tumor growth rate, etc. In addition, efficacy of an agent can be measured by a decrease in circulating MIC peptides or fragments thereof in a subject being treated with an agent comprising an antibody or antigen-binding portion thereof as described herein or a nucleic acid encoding an antibody or antigen-binding portion thereof as described herein.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### Examples

Materials and Methods

Identification of sMIC/MIC negative cancers: sMIC-negative tumors can be identified by assaying serum levels sMICA or sMICB using standard MICA or MICB detection ELISA. MIC-negative subjects should contain no detectable sMIC beyond background noise. For tumors that the biopsy is available, negative for MIC expression can also be selected or confirmed by immunohistochemistry showing no cross-reactivity with an anti-MIC antibody.

Peptide-binding region of the non-blocking anti-sMIC/MIC antibody D4H3: Using chemical cross-linking, High-Mass MALDI mass spectrometry and nLC-Orbitrap mass spectrometry the interaction interface between the Antigen and the antibody Ab-D4H3 was characterized. Results indicated that Ab-D4H3 binds to two regions of the antigen with the following amino acids on Antigen: 68, 72, 75, 77 and 206, 207 and 209. These regions are not in competition with NKG2D binding region in the alpha-1 and alpha-2 domain of MIC (Li et cl., Nat Immunol. 2001 May; 2(5):443-51., the disclosure of which is incorporated herein by reference in its entirety).

Generation of sMIC/anti-sMIC complex: Complex was formed by mixing sMIC with the anti-sMIC antibody at room temperature or 37° C. (molar ratio 1:1 or 2:1). Complex can also be formed by linking sMIC to the anti-sMIC antibody heavy chain or light chain with a polylinker.

### Example 1—sMIC/Anti-sMIC Complex Augments CD3/TCR-Mediated CD8 T Cell Activation In Vitro

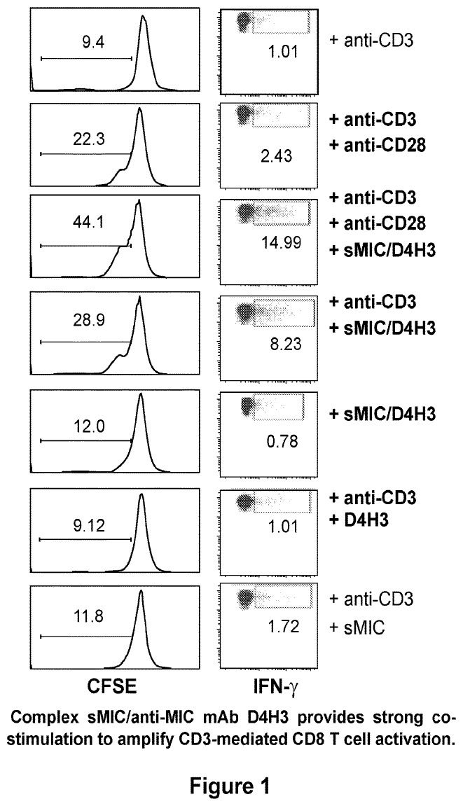

PBMCs from normal donors were stimulated in the presence or absence of plate-bound CD3 in the presence of sMIC, anti-sMIC antibody (e.g, D4H3) or the complex of sMIC/anti-sMIC mAb, for 3 days and assayed CD8 T cell IFNγ production by intracellular staining. Soluble anti-CD28 stimulation was used as a positive control. As shown in FIG. 1, similar to anti-CD28 stimulation, sMIC/anti-MIC complex activated CD8 T cells upon CD3 ligation, suggesting a co-stimulatory effect of the sMIC/anti-MIC complex. Neither sMIC nor anti-MIC mAb alone had similar effect. Moreover, sMIC/anti-MIC and anti-CD28 produced an additive effect to amplify CD3-mediated CD8 T cell activation (FIG. 1). Moreover, carboxyfluorescein diacetate succinimidyl ester (CFSE) dilution assay demonstrated that sMIC/anti-MIC co-stimulation also enhanced CD8 T cell proliferation (FIG. 1).

In another example, PBMCs were stimulated in the presence or absence of plate-bound CD3 in the presence of sMIC, anti-sMIC antibody (e.g, N04) or the complex of sMIC/NO4, for 48 hours followed by assaying CD8 T cell surface NKG2D expression and IFNγ production by intracellular staining. Similarly, anti-CD28 agonist antibody stimulation was used as a positive control for CD8 T cell co-stimulation. FIG. 2 demonstrates that complex stimulation amplified CD3/TCR activation as measured by IFNγ production and proliferation by CFSE dilution assay. Interestingly, CD3 and sMIC/NO4 complex stimulation increased CD8 T cell surface NKG2D expression as compared to CD3 or CD3/anti-CD28 stimulation. In summary, the data provided in this Example demonstrated that sMIC/anti-sMIC complex co-stimulation is non-redundant of anti-Cd28 co-stimulation.

### Example 2—sMIC/Anti-sMIC Complex Amplifies Antigen-Specific CD8 T Cells Responses

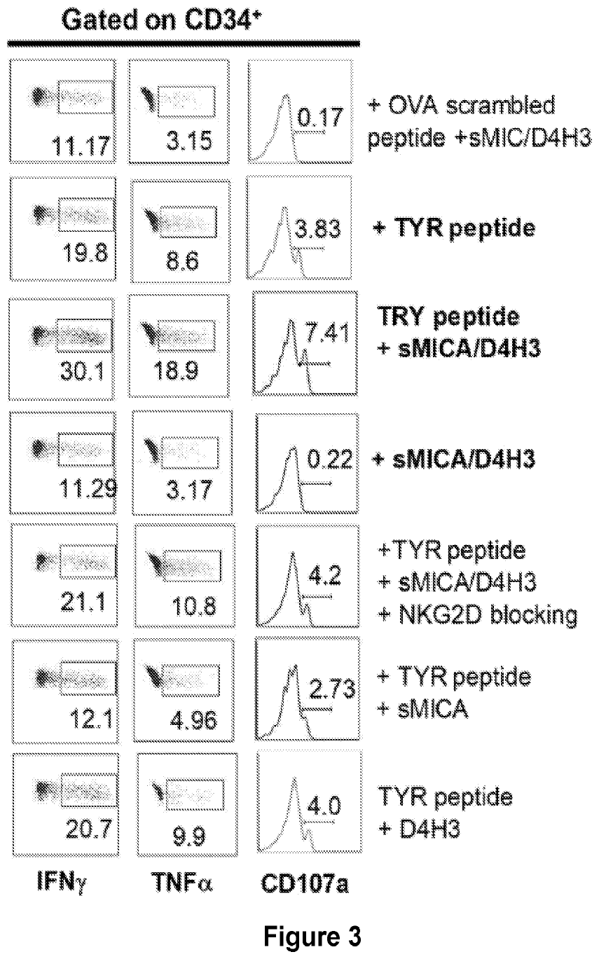

TIL1383I cells (engineered to express CD34 as a reporter) were cultured with HLA-A2⁺ surrogate T2-A2 antigen presenting cells (APC), in the presence or absence of sMIC(A)/D4H3 complex or tyrosinase peptide₃₆₉₋₃₇₇. After overnight culture, TIL13831 activation by intracellular staining for IFNγ, TNFα, and CD107a (degranulation) was assessed. As shown in FIG. 3, the sMIC/D4H3 complex remarkably enhanced TIL13831 to HLA-A2-restricted tyrosinase peptide stimulation. sMIC/D4H3 complex together with the scrambled OVA peptide did not stimulate TIL13831. Blocking NKG2D with mAb M585 abolished the co-stimulatory effect of sMIC/D4H3.

### Example 3—Therapy with sMIC/D4H3 Complex Inhibits the Growth of MIC-Negative Tumors

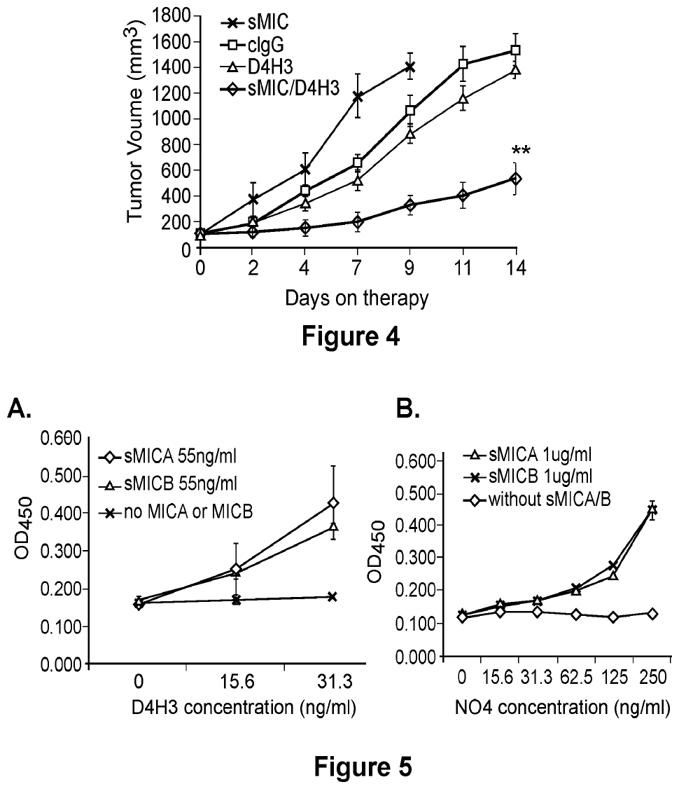

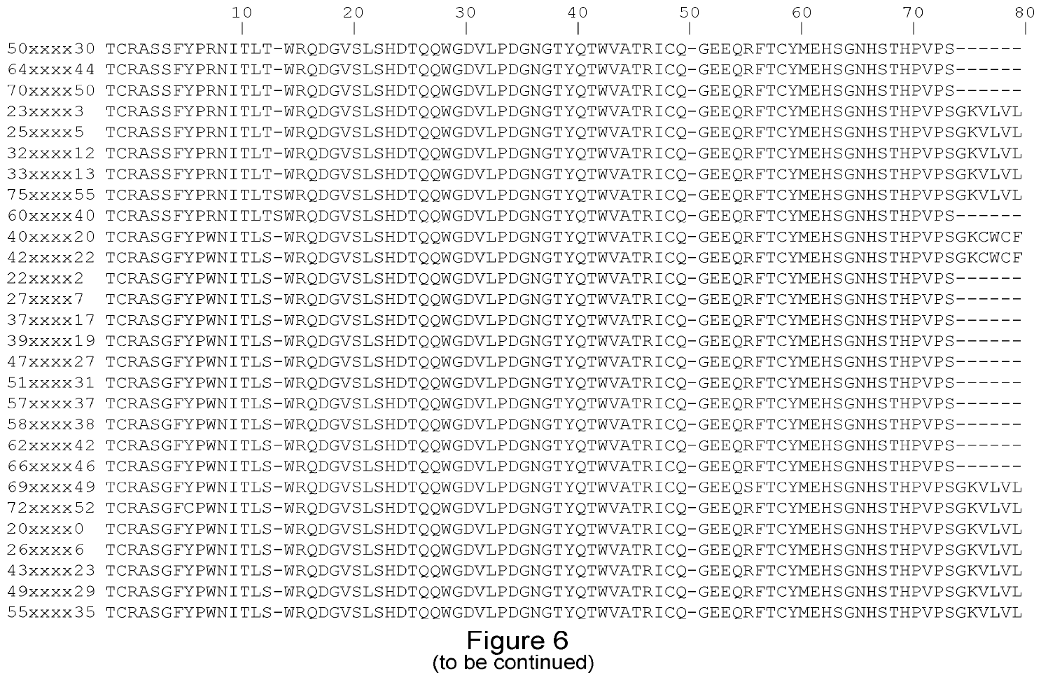

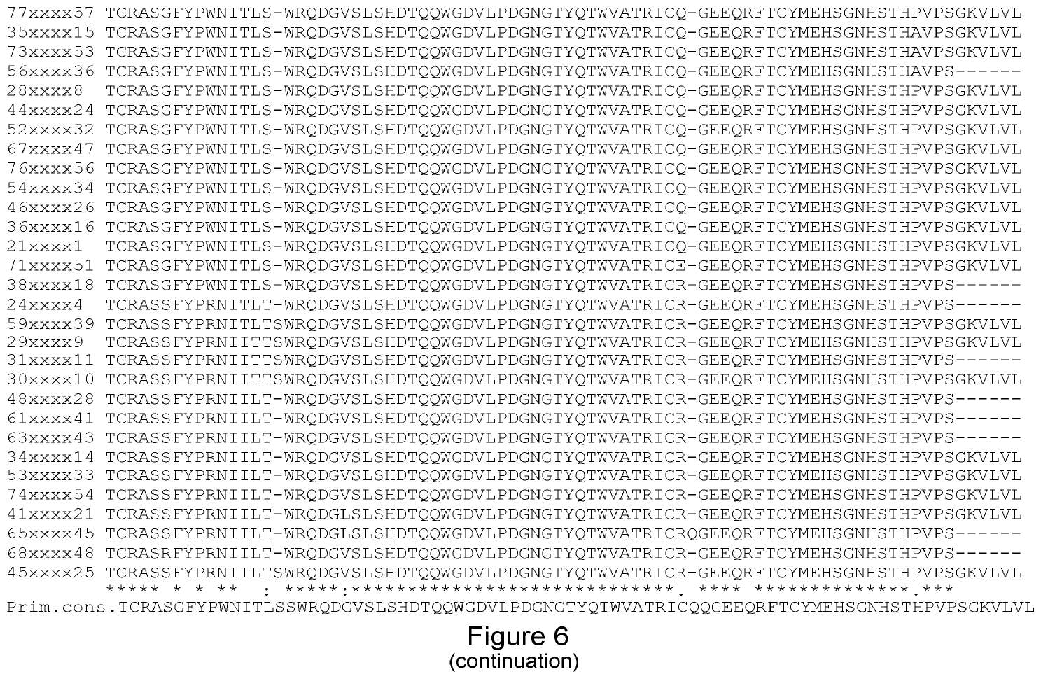

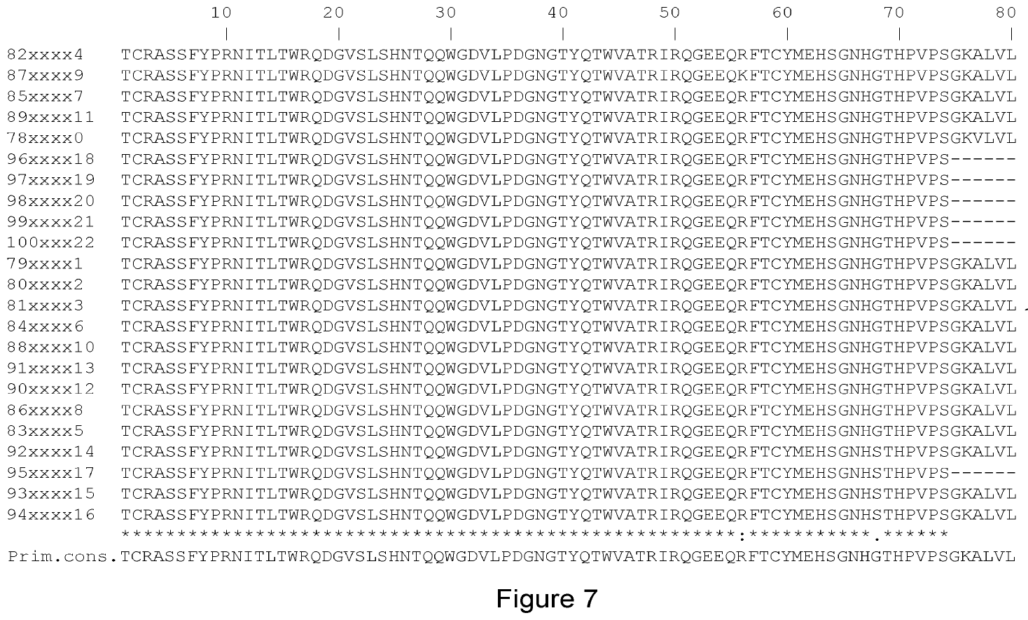

MIC^{negative} MC38 colon tumor cells were implanted into cohorts of syngeneic B6/MICB male mice. When tumors reached the volume of approximately 100 mm³ in size, animals were treated with control IgG, recombinant rsMIC, D4H3 or sMIC/D4H3 complex, respectively, individually or in combination by intraperitoneal administration twice a week at the dose of 4 mg/kg. As shown in FIG. 4, the sMIC/D4H3 complex significantly inhibited tumor growth. Complex therapy evoked antigen-specific immune response.

### Example 4—Detection of MIC/Anti-sMIC Antibody Binding to Receptor NKG2D by ELISA

An ELISA method was used to demonstrate that the complex composed of sMIC and a non-blocking anti-sMIC antibody (e.g., NO4 or D4H3) binds to the receptor NKG2D in order to activate NKG2D-mediated co-stimulatory signaling.

FIG. 5 demonstrates that the complex binds to NKG2D by ELISA assay. In this assay, recombinant soluble human NKG2D (rs-hNKG2D) was immobilized to a 96-well plate overnight, after washing to remove unbound rs-hNKG2D, given concentration of recombinant sMICA (source: R&D systems) or recombinant MICB-His (source: Fisher) complexed with various concentrations of the anti-sMIC monoclonal antibody D4H3 (FIG. 5A) or NO4 (FIG. 5B) (mouse IgG) was added. The binding of the complex to rs-hNKG2D was detected with an HRP-conjugated goat anti-mouse antibody.

### Example 5—sMIC/D4H3 Complex Therapy Rapidly Clears LCMV Viral Infection

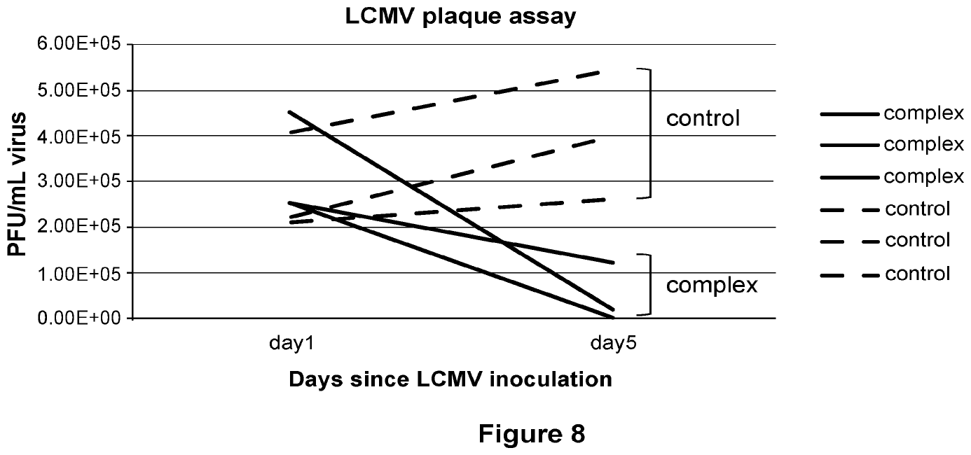

Cohorts of C57BL/6 mice were intravenously (i.v.) inoculated with 2×10⁶ PFU LCMV (Lymphocytic choriomeningitis virus) Armstrong strain. sMIC/D4H3 complex (6 mg/Kg) or control PBS were given (i.p. injection) to mice: Day 1 and Day 3 after viral inoculation. Blood samples were taken from mice at day 1 and day 5 post virus inoculation. Serum viral load were assayed with plaque assay (as previously described in Welsh and Seedhom in Curr Protoc Microbiol. 2008 February; CHAPTER: Unit-15A.1). As shown in FIG. 8, LCMV titres were significantly reduced in mice received that the sMIC/D4H3 complex therapy.

Any journal articles or patent documents referenced herein are incorporated herein by reference in their entireties.

What is claimed is:

1. A Natural Killer Group 2D (NKG2D) agonist complex comprising a soluble MHC I Chain-related molecule (sMIC) and a nonblocking sMIC-neutralizing antibody.

2. The complex claim 1, wherein the non-blocking antibody comprises CDRs set forth in SEQ ID NOs: 4-9.

3. The complex of claim 1 or claim 2, wherein the non-blocking antibody comprises a light chain variable region set for in the SEQ ID NO: 11.

4. The complex of any one of claims 1-3, wherein the non-blocking antibody comprises a heavy chain variable region set for in the SEQ ID NO: 10.

5. The complex of claim 1, wherein the non-blocking antibody comprises CDRs set forth in SEQ ID NOs: 12-17.

6. The complex of claim 1 or claim 5, wherein the non-blocking antibody comprises a light chain variable region set for in the SEQ ID NO: 19.

7. The complex of claim 1, claim 5 or claim 6, wherein the non-blocking antibody comprises a heavy chain variable region set for in the SEQ ID NO: 18.

8. The complex of any one of claims 1-7, wherein the sMIC is sMICA.

9. The complex of claim 1-7, wherein the sMIC is sMICB.

10. The complex of any one of claims 1-8, wherein the sMICA comprises an amino acid sequence set forth one of SEQ ID NOs: 1 and 20-77.

11. The complex of any one of claims 1-7 and 9, wherein the sMICB comprises an amino acid sequence set forth one of SEQ ID NOs: 2 and 78-100.

12. A composition comprising the complex of any one of claims 1-11 and a pharmaceutically acceptable carrier, diluent or adjuvant.

13. A method of activating CD8 T cells in a subject in need thereof, comprising administering to the subject the complex of any one of claims 1-11.

14. The method of claim 13, wherein the subject is suffering from cancer.

15. The method of claim 14, wherein the cancer is basal cell carcinoma, biliary tract cancer, bladder cancer, bone cancer, brain and CNS cancer, breast cancer, cancer of the peritoneum, cervical cancer; choriocarcinoma, colon and rectum cancer, connective tissue cancer, cancer of the digestive system, endometrial cancer, esophageal cancer, eye cancer, cancer of the head and neck; gastric cancer, gastrointestinal cancer; glioblastoma (GBM), hepatic carcinoma, hepatoma, intra-epithelial neoplasm, renal cancer, larynx cancer, leukemia, liver cancer, lung cancer, small-cell lung cancer, non-small cell lung cancer, adenocarcinoma of the lung, squamous carcinoma of the lung, lymphoma including Hodgkin's and non-Hodgkin's lymphoma, melanoma, myeloma, neuroblastoma, oral cavity cancer (e.g., lip, tongue, mouth, and pharynx), ovarian cancer, pancreatic cancer, prostate cancer, retinoblastoma, rhabdomyosarcoma, rectal cancer, cancer of the respiratory system, salivary gland carcinoma, sarcoma, skin cancer, squamous cell cancer, stomach cancer, testicular cancer, thyroid cancer, uterine or endometrial cancer, cancer of the urinary system, vulval cancer, B-cell lymphoma (including low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, mantle cell lymphoma, AIDS-related lymphoma, Waldenstrom's Macroglobulinemia, chronic lymphocytic leukemia (CLL), acute lymphoblastic leukemia (ALL), Hairy cell leukemia, chronic myeloblastic leukemia, and post-transplant lymphoproliferative disorder (PTLD), as well as abnormal vascular proliferation associated with phakomatoses, edema or Meigs' syndrome.

16. The method of any one of claims 13-15, wherein the subject is suffering from a MHC I Chain-related molecule (MIC)-negative cancer.

17. The method of claim 13, wherein the subject is suffering from a viral infection.

18. The method of claim 17, wherein the viral infection is caused by a DNA Virus (e.g., Herpes Viruses such as Herpes Simplex virus, Epstein-Barr virus, Cytomegalovirus; Pox viruses such as Variola (small pox) virus; Hepadnaviruses (e.g, Hepatitis B virus); Papilloma viruses; Adenovinises); RNA Viruses (e.g., HIV I, II; HTLV I, II; Poliovirus; Hepatitis A; coronoviruses, such as sudden acute respiratory syndrome (SARS); Orthomyxoviruses (e.g., Influenza viruses); Paramyxoviruses (e.g., Measles virus); Rabies virus; Hepatitis C virus), Flaviviruses, Influenza viruses; caliciviruses; or rabies viruses, rinderpest viruses and Arena virus.

19. The method of claim 17, wherein the viral infection is caused by lymphocytic choriomeningitis (LCMV).

20. A method of viral infection in a subject in need thereof, comprising administering to the subject the complex of any one of claims 1-11.

21. A method of treating cancer in a subject in need thereof, comprising administering to the subject the complex of any one of claims 1-11.

22. The method of claim 21, wherein the cancer is basal cell carcinoma, biliary tract cancer, bladder cancer, bone cancer, brain and CNS cancer, breast cancer, cancer of the peritoneum, cervical cancer; choriocarcinoma, colon and rectum cancer, connective tissue cancer, cancer of the digestive system, endometrial cancer, esophageal cancer, eye cancer, cancer of the head and neck; gastric cancer, gastrointestinal cancer; glioblastoma (GBM), hepatic carcinoma, hepatoma, intra-epithelial neoplasm, renal cancer, larynx cancer, leukemia, liver cancer, lung cancer, small-cell lung cancer, non-small cell lung cancer, adenocarcinoma of the lung, squamous carcinoma of the lung, lymphoma including Hodgkin's and non-Hodgkin's lymphoma, melanoma, myeloma, neuroblastoma, oral cavity cancer (e.g., lip, tongue, mouth, and pharynx), ovarian cancer, pancreatic cancer, prostate cancer, retinoblastoma, rhabdomyosarcoma, rectal cancer, cancer of the respiratory system, salivary gland carcinoma, sarcoma, skin cancer, squamous cell cancer, stomach cancer, testicular cancer, thyroid cancer, uterine or endometrial cancer, cancer of the urinary system, vulval cancer, B-cell lymphoma (including low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, mantle cell lymphoma, AIDS-related lymphoma, Waldenstrom's Macroglobulinemia, chronic lymphocytic leukemia (CLL), acute lymphoblastic leukemia (ALL), Hairy cell leukemia, chronic myeloblastic leukemia, and post-transplant lymphoproliferative disorder (PTLD).

23. The method of claim 21 or claim 22, wherein the subject is suffering from a MHC I Chain-related molecule (MIC)-negative cancer.

24. The method of any one of claims 12-16 and 21-23, further comprising administering an immune checkpoint inhibitor to the subject.

25. The method of claim 24, wherein the immune checkpoint inhibitor is MGA27, ipilimumab, pembrolizumab, nivolumab, atezolizumab, IMP321, IPH2101, tremelimumab, pidilizumab, MPDL3280A, MEDI4736, MSB0010718C, AUNP12, avelumab, durvalumab, and TSR-022.


SEQUENCE LISTING (100 sequences)

SEQ ID NO: 1 - protein, 383 aa, Homo Sapiens
MGLGPVFLLLAGIFPFAPPGAAAEPHSLRYNLTVLSWDGSVQSGFLTEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETKEWTMPQSSRAQTLAMNVRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPSGKVLVLQSHWQTFHVSAVAAAAIFVIIIFYVRCCKKKTSAAEGPELVSLQVLDQHPVGTSDHRDATQLGFQPLMSDLGSTGSTEGA

SEQ ID NO: 2 - protein, 383 aa, Homo Sapiens
MGLGRVLLFLAVAFPFAPPAAAAEPHSLRYNLMVLSQDGSVQSGFLAEGHLDGQPFLRYDRQKRRAKPQGQWAENVLGAKTWDTETEDLTENGQDLRRTLTHIKDQKGGLHSLQEIRVCEIHEDSSTRGSRHFYYDGELFLSQNLETQESTVPQSSRAQTLAMNVTNFWKEDAMKTKTHYRAMQADCLQKLQRYLKSGVAIRRTVPPMVNVTCSEVSEGNITVTCRASSFYPRNITLTWRQDGVSLSHNTQQWGDVLPDGNGTYQTWVATRIRQGEEQRFTCYMEHSGNHGTHPVPSGKALVLQSQRTDFPYVSAAMPCFVIIIILCVPCCKKKTSAAEGPELVSLQVLDQHPVGTGDHRDAAQLGFQPLMSATGSTGSTEGT

SEQ ID NO: 3 - protein, 274 aa, Artificial Sequence
  Synthetic Polypeptide
EPHSLRYNLTVLSWDGSVQSGFLAEVHLDGQPFLRYDRQKCRAKPQGQWAEDVLGNKTWDRETRDLTGNGKDLRMTLAHIKDQKEGLHSLQEIRVCEIHEDNSTRSSQHFYYDGELFLSQNVETEEWTVPQSSRAQTLAMNVRNFLKEDAMKTKTHYHAMHADCLQELRRYLESSVVLRRRVPPMVNVTRSEALEGNITVTCGASSFYPRNITLTWRQDGVSLSHDTQQWGDVLPDGNGTYQTWVATRICQGEEQRFTCYMEHSGNHSTHPVPS

SEQ ID NO: 4 - protein, 11 aa, Artificial Sequence
  Synthetic Polypeptide
QSIVHSNGDTY

SEQ ID NO: 5 - protein, 3 aa, Artificial Sequence
  Synthetic Polypeptide
KVS

SEQ ID NO: 6 - protein, 9 aa, Artificial Sequence
  Synthetic Polypeptide
FQGSHFPWT

SEQ ID NO: 7 - protein, 8 aa, Artificial Sequence
  Synthetic Polypeptide
GFTFSYFA

SEQ ID NO: 8 - protein, 8 aa, Artificial Sequence
  Synthetic Polypeptide
ISRGGDYI

SEQ ID NO: 9 - protein, 14 aa, Artificial Sequence
  Synthetic Polypeptide
ACISTVVEGAWLAY

SEQ ID NO: 10 - protein, 121 aa, Artificial Sequence
  Synthetic Polypeptide
EVMLVESGGGLVKPGGSLRLSCAASGFTFSYFAMSWVRQTPEKRLEWVATISRGGDYIYYPDSVKGRFTISRDNAKNTLYLQTSSLRSEDTAMYYCACISTVVEGAWLAYWGQGTLVTVSA

SEQ ID NO: 11 - protein, 112 aa, Artificial Sequence
  Synthetic Polypeptide
DVLMTQTPFSLPVSLGDQASISCRSSQSIVHSNGDTYLEWYLQKPGQSPKLLIYKVSNRFSGVPVRFSGSGSGTVFTLKISRVEAEDLGVYYCFQGSHFPWTFGGGTKLEIK

SEQ ID NO: 12 - protein, 7 aa, Artificial Sequence
  Synthetic Polypeptide
SSISSNY

SEQ ID NO: 13 - protein, 7 aa, Artificial Sequence
  Synthetic Polypeptide
SSISSNY

SEQ ID NO: 14 - protein, 9 aa, Artificial Sequence
  Synthetic Polypeptide
QQAITIPFT

SEQ ID NO: 15 - protein, 9 aa, Artificial Sequence
  Synthetic Polypeptide
GYSITSDYA

SEQ ID NO: 16 - protein, 7 aa, Artificial Sequence
  Synthetic Polypeptide
ISYSGST

SEQ ID NO: 17 - protein, 8 aa, Artificial Sequence
  Synthetic Polypeptide
ARGWAMDY

SEQ ID NO: 18 - protein, 116 aa, Artificial Sequence
  Synthetic Polypeptide
SDVQLQESGPGLVKPSQSLSLTCTVTGYSITSDYAWNWIRQFPGNKLDWMGYISYSGSTRYNPSLTSRISITRDTSKNQFFLQLNSVTTEDTATYYCARGWAMDYWGQGTSVTVSS

SEQ ID NO: 19 - protein, 108 aa, Artificial Sequence
  Synthetic Polypeptide
EIVLTQSPTTMAASPGEKITITCSASSSISSNYLHWYQQKPGFSPKLLIYRTSNLASGVPARFSGSGSGTSYSLTIGTMEAEDVATYYCQQAITIPFTFGSGTKLEIK

SEQ ID NO: 20 - protein, 80 aa, Homo sapiens
TCRASGFYPWNITLSWRQDGVSLSHDTQQWGDVLPDGNGTYQTWVATRICQGEEQRFTCYMEHSGNHSTHPVPSGKVLVL

SEQ ID NO: 21 - protein, 80 aa, Homo sapiens; identical to SEQ ID NO: 20

SEQ ID NO: 22 - protein, 74 aa, Homo sapiens
TCRASGFYPWNITLSWRQDGVSLSHDTQQWGDVLPDGNGTYQTWVATRICQGEEQRFTCYMEHSGNHSTHPVPS

SEQ ID NO: 23 - protein, 80 aa, Homo sapiens
TCRASSFYPRNITLTWRQDGVSLSHDTQQWGDVLPDGNGTYQTWVATRICQGEEQRFTCYMEHSGNHSTHPVPSGKVLVL

SEQ ID NO: 24 - protein, 74 aa, Homo sapiens
TCRASSFYPRNITLTWRQDGVSLSHDTQQWGDVLPDGNGTYQTWVATRICRGEEQRFTCYMEHSGNHSTHPVPS

SEQ ID NO: 25 - protein, 80 aa, Homo sapiens; identical to SEQ ID NO: 23

SEQ ID NO: 26 - protein, 80 aa, Homo sapiens; identical to SEQ ID NO: 20

SEQ ID NO: 27 - protein, 74 aa, Homo sapiens; identical to SEQ ID NO: 22

SEQ ID NO: 28 - protein, 80 aa, Homo sapiens; identical to SEQ ID NO: 20

SEQ ID NO: 29 - protein, 81 aa, Homo sapiens
TCRASSFYPRNIITTSWRQDGVSLSHDTQQWGDVLPDGNGTYQTWVATRICRGEEQRFTCYMEHSGNHSTHPVPSGKVLVL

SEQ ID NO: 30 - protein, 81 aa, Homo sapiens; identical to SEQ ID NO: 29

SEQ ID NO: 31 - protein, 75 aa, Homo sapiens
TCRASSFYPRNIITTSWRQDGVSLSHDTQQWGDVLPDGNGTYQTWVATRICRGEEQRFTCYMEHSGNHSTHPVPS

SEQ ID NO: 32 - protein, 80 aa, Homo sapiens; identical to SEQ ID NO: 23

SEQ ID NO: 33 - protein, 80 aa, Homo sapiens; identical to SEQ ID NO: 23

SEQ ID NO: 34 - protein, 80 aa, Homo sapiens
TCRASSFYPRNIILTWRQDGVSLSHDTQQWGDVLPDGNGTYQTWVATRICRGEEQRFTCYMEHSGNHSTHPVPSGKVLVL

SEQ ID NO: 35 - protein, 80 aa, Homo sapiens
TCRASGFYPWNITLSWRQDGVSLSHDTQQWGDVLPDGNGTYQTWVATRICQGEEQRFTCYMEHSGNHSTHAVPSGKVLVL

SEQ ID NO: 36 - protein, 80 aa, Homo sapiens; identical to SEQ ID NO: 20

SEQ ID NO: 37 - protein, 74 aa, Homo sapiens; identical to SEQ ID NO: 22

SEQ ID NO: 38 - protein, 74 aa, Homo sapiens
TCRASGFYPWNITLSWRQDGVSLSHDTQQWGDVLPDGNGTYQTWVATRICRGEEQRFTCYMEHSGNHSTHPVPS

SEQ ID NO: 39 - protein, 74 aa, Homo sapiens; identical to SEQ ID NO: 22

SEQ ID NO: 40 - protein, 80 aa, Homo sapiens
TCRASGFYPWNITLSWRQDGVSLSHDTQQWGDVLPDGNGTYQTWVATRICQGEEQRFTCYMEHSGNHSTHPVPSGKCWCF

SEQ ID NO: 41 - protein, 80 aa, Homo sapiens
TCRASSFYPRNIILTWRQDGLSLSHDTQQWGDVLPDGNGTYQTWVATRICRGEEQRFTCYMEHSGNHSTHPVPSGKVLVL

SEQ ID NO: 42 - protein, 80 aa, Homo sapiens; identical to SEQ ID NO: 40

SEQ ID NO: 43 - protein, 80 aa, Homo sapiens; identical to SEQ ID NO: 20

SEQ ID NO: 44 - protein, 80 aa, Homo sapiens; identical to SEQ ID NO: 20

SEQ ID NO: 45 - protein, 81 aa, Homo sapiens
TCRASSFYPRNIILTSWRQDGVSLSHDTQQWGDVLPDGNGTYQTWVATRICRGEEQRFTCYMEHSGNHSTHPVPSGKVLVL

SEQ ID NO: 46 - protein, 80 aa, Homo sapiens; identical to SEQ ID NO: 20

SEQ ID NO: 47 - protein, 74 aa, Homo sapiens; identical to SEQ ID NO: 22

SEQ ID NO: 48 - protein, 74 aa, Homo sapiens
TCRASSFYPRNIILTWRQDGVSLSHDTQQWGDVLPDGNGTYQTWVATRICRGEEQRFTCYMEHSGNHSTHPVPS

SEQ ID NO: 49 - protein, 80 aa, Homo sapiens; identical to SEQ ID NO: 20

SEQ ID NO: 50 - protein, 74 aa, Homo sapiens
TCRASSFYPRNITLTWRQDGVSLSHDTQQWGDVLPDGNGTYQTWVATRICQGEEQRFTCYMEHSGNHSTHPVPS

SEQ ID NO: 51 - protein, 74 aa, Homo sapiens; identical to SEQ ID NO: 22

SEQ ID NO: 52 - protein, 80 aa, Homo sapiens; identical to SEQ ID NO: 20

SEQ ID NO: 53 - protein, 80 aa, Homo sapiens; identical to SEQ ID NO: 34

SEQ ID NO: 54 - protein, 80 aa, Homo sapiens; identical to SEQ ID NO: 20

SEQ ID NO: 55 - protein, 80 aa, Homo sapiens; identical to SEQ ID NO: 20

SEQ ID NO: 56 - protein, 74 aa, Homo sapiens
TCRASGFYPWNITLSWRQDGVSLSHDTQQWGDVLPDGNGTYQTWVATRICQGEEQRFTCYMEHSGNHSTHAVPS

SEQ ID NO: 57 - protein, 74 aa, Homo sapiens; identical to SEQ ID NO: 22

SEQ ID NO: 58 - protein, 74 aa, Homo sapiens; identical to SEQ ID NO: 22

SEQ ID NO: 59 - protein, 81 aa, Homo sapiens
TCRASSFYPRNITLTSWRQDGVSLSHDTQQWGDVLPDGNGTYQTWVATRICRGEEQRFTCYMEHSGNHSTHPVPSGKVLVL

SEQ ID NO: 60 - protein, 75 aa, Homo sapiens
TCRASSFYPRNITLTSWRQDGVSLSHDTQQWGDVLPDGNGTYQTWVATRICQGEEQRFTCYMEHSGNHSTHPVPS

SEQ ID NO: 61 - protein, 74 aa, Homo sapiens; identical to SEQ ID NO: 48

SEQ ID NO: 62 - protein, 74 aa, Homo sapiens; identical to SEQ ID NO: 22

SEQ ID NO: 63 - protein, 74 aa, Homo sapiens; identical to SEQ ID NO: 48

SEQ ID NO: 64 - protein, 74 aa, Homo sapiens; identical to SEQ ID NO: 50

SEQ ID NO: 65 - protein, 75 aa, Homo sapiens
TCRASSFYPRNIILTWRQDGLSLSHDTQQWGDVLPDGNGTYQTWVATRICRQGEEQRFTCYMEHSGNHSTHPVPS

SEQ ID NO: 66 - protein, 74 aa, Homo sapiens; identical to SEQ ID NO: 22

SEQ ID NO: 67 - protein, 80 aa, Homo sapiens; identical to SEQ ID NO: 20

SEQ ID NO: 68 - protein, 74 aa, Homo sapiens
TCRASRFYPRNIILTWRQDGVSLSHDTQQWGDVLPDGNGTYQTWVATRICRGEEQRFTCYMEHSGNHSTHPVPS

SEQ ID NO: 69 - protein, 80 aa, Homo sapiens
TCRASGFYPWNITLSWRQDGVSLSHDTQQWGDVLPDGNGTYQTWVATRICQGEEQSFTCYMEHSGNHSTHPVPSGKVLVL

SEQ ID NO: 70 - protein, 74 aa, Homo sapiens; identical to SEQ ID NO: 50

SEQ ID NO: 71 - protein, 80 aa, Homo sapiens
TCRASGFYPWNITLSWRQDGVSLSHDTQQWGDVLPDGNGTYQTWVATRICEGEEQRFTCYMEHSGNHSTHPVPSGKVLVL

SEQ ID NO: 72 - protein, 80 aa, Homo sapiens
TCRASGFCPWNITLSWRQDGVSLSHDTQQWGDVLPDGNGTYQTWVATRICQGEEQRFTCYMEHSGNHSTHPVPSGKVLVL

SEQ ID NO: 73 - protein, 80 aa, Homo sapiens; identical to SEQ ID NO: 35

SEQ ID NO: 74 - protein, 80 aa, Homo sapiens; identical to SEQ ID NO: 34

SEQ ID NO: 75 - protein, 81 aa, Homo sapiens
TCRASSFYPRNITLTSWRQDGVSLSHDTQQWGDVLPDGNGTYQTWVATRICQGEEQRFTCYMEHSGNHSTHPVPSGKVLVL

SEQ ID NO: 76 - protein, 80 aa, Homo sapiens; identical to SEQ ID NO: 20

SEQ ID NO: 77 - protein, 80 aa, Homo sapiens; identical to SEQ ID NO: 20

SEQ ID NO: 78 - protein, 80 aa, Homo sapiens
TCRASSFYPRNITLTWRQDGVSLSHNTQQWGDVLPDGNGTYQTWVATRIRQGEEQRFTCYMEHSGNHGTHPVPSGKVLVL

SEQ ID NO: 79 - protein, 80 aa, Homo sapiens
TCRASSFYPRNITLTWRQDGVSLSHNTQQWGDVLPDGNGTYQTWVATRIRQGEEQRFTCYMEHSGNHGTHPVPSGKALVL

SEQ ID NO: 80 - protein, 80 aa, Homo sapiens; identical to SEQ ID NO: 79

SEQ ID NO: 81 - protein, 80 aa, Homo sapiens; identical to SEQ ID NO: 79

SEQ ID NO: 82 - protein, 80 aa, Homo sapiens; identical to SEQ ID NO: 79

SEQ ID NO: 83 - protein, 80 aa, Homo sapiens; identical to SEQ ID NO: 79

SEQ ID NO: 84 - protein, 80 aa, Homo sapiens; identical to SEQ ID NO: 79

SEQ ID NO: 85 - protein, 80 aa, Homo sapiens; identical to SEQ ID NO: 79

SEQ ID NO: 86 - protein, 80 aa, Homo sapiens; identical to SEQ ID NO: 79

SEQ ID NO: 87 - protein, 80 aa, Homo sapiens
TCRASSFYPRNITLTWRQDGVSLSHNTQQWGDVLPDGNGTYQTWVATRIRQGEEQKFTCYMEHSGNHGTHPVPSGKALVL

SEQ ID NO: 88 - protein, 80 aa, Homo sapiens; identical to SEQ ID NO: 79

SEQ ID NO: 89 - protein, 80 aa, Homo sapiens; identical to SEQ ID NO: 79

SEQ ID NO: 90 - protein, 80 aa, Homo sapiens; identical to SEQ ID NO: 79

SEQ ID NO: 91 - protein, 80 aa, Homo sapiens; identical to SEQ ID NO: 79

SEQ ID NO: 92 - protein, 80 aa, Homo sapiens
TCRASSFYPRNITLTWRQDGVSLSHNTQQWGDVLPDGNGTYQTWVATRIRQGEEQRFTCYMEHSGNHSTHPVPSGKALVL

SEQ ID NO: 93 - protein, 80 aa, Homo sapiens; identical to SEQ ID NO: 92

SEQ ID NO: 94 - protein, 80 aa, Homo sapiens; identical to SEQ ID NO: 92

SEQ ID NO: 95 - protein, 74 aa, Homo sapiens
TCRASSFYPRNITLTWRQDGVSLSHNTQQWGDVLPDGNGTYQTWVATRIRQGEEQRFTCYMEHSGNHSTHPVPS

SEQ ID NO: 96 - protein, 74 aa, Homo sapiens
TCRASSFYPRNITLTWRQDGVSLSHNTQQWGDVLPDGNGTYQTWVATRIRQGEEQRFTCYMEHSGNHGTHPVPS

SEQ ID NO: 97 - protein, 74 aa, Homo sapiens; identical to SEQ ID NO: 96

SEQ ID NO: 98 - protein, 74 aa, Homo sapiens; identical to SEQ ID NO: 96

SEQ ID NO: 99 - protein, 74 aa, Homo sapiens; identical to SEQ ID NO: 96

SEQ ID NO: 100 - protein, 74 aa, Homo sapiens; identical to SEQ ID NO: 96